(19) United States
(12) Patent Application Publication
(10) Pub. No.: US 2026/0248978 A1
(43) Pub. Date: Aug. 27, 2026

(54) SYSTEM AND DEVICE FOR REFLECTING ULTRAVIOLET RADIATION

(71) Applicant: Pathogen Reduction Solutions Limited, Horley, Surrey (GB)

(72) Inventors: Robert McCullough, Sausalito, CA (US); Ian Sinclair, Horley, Surrey (GB); Julia Martin, Horley, Surrey (GB)

(21) Appl. No.: 18/870,423

(22) PCT Filed: May 31, 2023
(86) PCT No.: PCT/GB2023/051437

§ 371 Date: Nov. 28, 2024

(30) Foreign Application Priority Data
May 31, 2022 (GB) 2208032.9
Mar. 7, 2023 (GB) 2303313.7

(51) Int. Cl. A61L 9/20

(52) U.S. Cl. CPC A61L 9/20

(57) ABSTRACT

The present disclosure relates to an air handling system, comprising: at least one source of ultraviolet radiation; one or more radiation-attenuating devices for attenuating ultraviolet radiation; and a reflective surface configured to reflect incident ultraviolet radiation away from the one or more radiation-attenuating devices.

### FIELD

The present disclosure relates to a device for reflecting ultraviolet (UV) radiation. In particular, the present disclosure relates to a device for reflecting UV radiation emitted by an air cleaning system. The present disclosure also relates to an air handling system for reflecting UV radiation, an air handling system comprising a device for reflecting UV radiation, and a method of reflecting UV radiation.

### BACKGROUND

Air handling systems such as heating, ventilation and air conditioning (HVAC) systems are used to supply air to buildings and other locations. Typically, these air handling systems recirculate recycled air within the building. The recirculation of recycled air can involve recirculation of pathogens, if one or more occupants of the building are infected with a particular disease.

UV radiation (e.g. UVC radiation) can be used to irradiate pathogens present in airflow through an air handling system, thereby damaging the pathogens and rendering them inactive. By using UV radiation to “clean” the airflow in this way, air that is potentially contaminated can be recirculated in the air handling system. An alternative to cleaning the airflow would be to increase the proportion of non-recycled air used in the air handling system. Existing air handling systems are designed to operate with a maximum non-recycled air fraction of approximately 30%. Running these existing systems at 100% non-recycled air would result in insufficient heating or cooling of the air supply, causing discomfort for the building's occupants and potentially resulting in an unusable working environment. Modifying these existing systems to increase the heating or cooling capacity of the air supply systems would incur significant cost owing to the increase in energy usage, and would result in substantially higher running costs for the building.

When using a source UV radiation to irradiate pathogens present in airflow through an air handling system, a particular volume of air is only irradiated by the UV radiation for a short period of time, before it continues its passage through the air handling system. To compensate for this, the dosage of the UV radiation can be increased in order to ensure the inactivation of pathogens in the airflow. It will be appreciated, however, that increasing the dosage increases the amount of energy consumed when cleaning the airflow. It is therefore desirable to increase the efficiency of systems that utilise UV radiation to inactivate airborne pathogens.

### SUMMARY

This summary introduces concepts that are described in more detail in the detailed description. It should not be used to identify essential features of the claimed subject matter, nor to limit the scope of the claimed subject matter.

According to a first aspect of the present disclosure, there is provided a device for reflecting ultraviolet radiation in an air handling system, the device comprising: a first end and a second end; a plurality of elongate flow passages, wherein each of the elongate flow passages is configured to permit airflow through the elongate flow passage; and one or more reflective surfaces configured to reflect incident ultraviolet radiation; wherein when the device is positioned in the air handling system such that the first end of the device is closer to a source of ultraviolet radiation than the second end of the device, the one or more reflective surfaces are configured to reflect ultraviolet radiation from the ultraviolet radiation source away from the second end of the device.

The device increases the energy flux density in the region of the ultraviolet radiation source, as a result of the one or more reflective surfaces that reflect incident ultraviolet radiation away from the second end of the device (i.e. back towards the ultraviolet radiation source). The reflection of ultraviolet radiation back towards the source of ultraviolet radiation means that the power requirement to provide a given energy flux density in the vicinity of the source of ultraviolet radiation is reduced. The given energy flux density may be the energy flux density required to provide a desired level of inactivation of one or more types of pathogen within the airflow. The device reduces the power requirement to provide the given energy flux density while minimising the pressure drop to airflow within the air handling system.

The one or more reflective surfaces may be retroreflective to incident ultraviolet radiation. Using a retroreflective surface ensures that the incident radiation is reflected back to where it originated from. This means that the one or more reflective surfaces can be provided on the walls of the elongate flow passages, which minimises obstruction of airflow.

Each of the plurality of elongate flow passages may comprise one or more internal walls. The one or more reflective surfaces may be disposed on at least a portion of at least one of the one or more internal walls of each of the plurality of elongate flow passages. This positioning of the one or more reflective surfaces minimises the impact on airflow resulting from implementation of the one or more reflective surfaces. The one or more reflective surfaces may extend across all internal walls of each of the plurality of elongate flow passages. In other words, all surfaces of all internal walls may be reflective (optionally retroreflective) to incident ultraviolet radiation.

Each of the plurality of elongate flow passages may comprise a first flow passage portion and a second flow passage portion. The first flow passage portion of each elongate flow passage may be located closer to the first end of the device than the second flow passage portion. The one or more reflective surfaces may be disposed on at least one of the one or more internal walls of the first flow passage portion of each elongate flow passage. This positioning of the one or more reflective surfaces optimises the reflection of radiation that is incident on internal walls of the elongate flow passages. The first flow passage portion of each elongate flow passage may be located adjacent to the first end of the device. This positioning is preferable because retroreflective surfaces are more effective at reflecting incident radiation when the angle of incidence is close to normal.

The length of the first flow passage portion of each elongate flow passage may be between 10% and 40% of the length of the respective elongate flow passage. These values are preferable because retroreflective surfaces are more effective at reflecting incident radiation when the angle of incidence is close to normal.

At least a portion of at least one of the one or more internal walls of each of the plurality of elongate flow passages may be configured to attenuate incident ultraviolet radiation. Accordingly, the device allows ultraviolet radiation to be attenuated to a particular level, which may be a safe exposure level for human operators, or a level at which degradation of components within the air handling system is reduced or eliminated. At the same time, the use of elongate flow passages allows air to flow through the device. The device therefore attenuates ultraviolet radiation while minimising impedance to air flow through the air handling system.

The at least a portion of the at least one of the one or more internal walls of each elongate flow passage may comprise a coating configured to absorb a proportion of incident ultraviolet radiation. More specifically, the coating may be configured to absorb a proportion of incident UVC radiation having a wavelength of between about 200 nm and 280 nm, more preferably between 210 nm and 260 nm, and even more preferably about 222 nm or about 254 nm.

The coating may be configured to reflect less than 60% of incident UVC radiation, preferably less than 50% of incident UVC radiation, more preferably less than 40% of incident UVC radiation, more preferably less than 30% of incident UVC radiation, more preferably less than 20% of incident UVC radiation, more preferably less than 10% of incident UVC radiation, and most preferably less than 5% of incident UVC radiation. The proportions of reflected UVC radiation given in the foregoing list provide progressively lower amounts of radiation reflected by the coating on the internal walls of the elongate flow passages, thereby providing progressively higher attenuations of ultraviolet radiation.

The coating may comprise one or more coats of black paint. More preferably, the coating may comprise two or more coats of black paint, and more preferably three or more coats of black paint. A black surface absorbs incident ultraviolet radiation, thereby attenuating the ultraviolet radiation. Using two or more coats of black paint provides increased attenuation of ultraviolet radiation when compared with a single coat of black paint. Attenuation is further increased by using three or more coats of black paint.

The second flow passage portion of each elongate flow passage may be configured to attenuate incident ultraviolet radiation. Accordingly, each elongate flow passage reflects incident radiation at certain angles of incidence, while attenuating incident radiation at other angles of incidence.

The device may be arranged for insertion in a ducting section of the air handling system such that any airflow through the ducting section flows through the elongate flow passages of the device. In this way, any air or radiation can only pass through the ducting section via an elongate flow passage.

The plurality of elongate flow passages may be arranged such that, collectively, the plurality of elongate flow passages has a substantially square or rectangular cross-section. This allows the device to be easily mounted in an air handing system that includes ducts with similar cross-sections.

Each of the plurality of elongate flow passages may be configured to permit air to flow substantially unimpeded through the elongate flow passage. Minimising the impedance to air flowing through the ducting section reduces the pressure drop in the air handling system resulting from including the device in the ducting section.

Each of the plurality of elongate flow passages may be straight. This minimises the resistance to air flowing through the elongate flow passage from the inlet to the outlet. In turn, this minimises the pressure drop within the air handling system as a result of implementing the device in the ducting section.

Each of the elongate flow passages may have a length that is significantly greater than a diameter of the flow passage. For example, the length may be four or more times greater than the diameter.

Each of the plurality of elongate flow passages may have an aspect ratio calculated by dividing a length of the elongate flow passage by a diameter of the elongate flow passage. The aspect ratio of each of the plurality of elongate flow passages may be greater than or equal to 4. An aspect ratio of greater than or equal to 4 means that ultraviolet photons are likely to reflect off the internal walls of the elongate flow passage. In addition, an aspect ratio of greater than or equal to 4 provides a low range of angles over which a photon can pass through the elongate flow passage without colliding with the internal walls.

The aspect ratio of each of the plurality of elongate flow passages may be less than or equal to 50. An aspect ratio of less than 50 reduces the boundary layer effects through each of the elongate flow passages, thereby reducing the pressure drop resulting from implementation of the device in the ducting section. The aspect ratio of each of the plurality of elongate flow passages may be less than or equal to 20. An aspect ratio of less than 20 further reduces the boundary layer effects through each of the elongate flow passages, thereby further reducing the pressure drop resulting from implementation of the ducting section.

The one or more walls of each of the plurality of elongate flow passages may be formed of aluminium. Aluminium is not degraded by ultraviolet radiation, meaning that the elongate flow passages are not damaged by exposure to the ultraviolet radiation.

Each of the plurality of elongate flow passages may have a hexagonal cross-section. Using a hexagonal cross-section allows the elongate flow passages to be easily fabricated using a honeycomb structure.

The plurality of elongate flow passages may be defined by a honeycomb structure of the device. The honeycomb structure helps to damp out turbulence in the airflow, which can provide beneficial effects downstream, such as reduced mixing and reduced frictional pressure drop.

According to a second aspect of the present disclosure, there is provided an air handling system comprising: an air cleaning system comprising: a ducting section comprising an inlet and an outlet; and at least one source of ultraviolet radiation arranged to emit ultraviolet radiation into an interior volume of the ducting section, the interior volume being between the inlet and the outlet; and a device according to the first aspect, wherein the device is located at one of the inlet and the outlet of the ducting section, wherein the device is configured to reflect ultraviolet radiation towards the interior volume.

The device increases the energy flux density within the interior volume of the air cleaning system, as a result of the one or more reflective surfaces that reflect incident ultraviolet radiation towards the interior volume. The reflection of ultraviolet radiation towards interior volume means that the power requirement to provide a given energy flux density within the interior volume is reduced. The device reduces the power requirement to provide the given energy flux density within the interior volume while minimising the pressure drop to airflow within the air handling system.

The device may be a first device. The air handling system may comprise a second device according to the first aspect. The second device may be located at the other one of the inlet and the outlet of the ducting section. Implementing two devices provides increased reflection of ultraviolet radiation into the interior volume of the air cleaning system, thereby providing increased energy flux density within the interior volume, and consequently further reducing the electrical power needed to provide an energy flux density associated with a desired inactivation ratio.

The ducting section may comprise one or more walls defining a cross-section of the ducting section. The device may extend across the entire cross-section of the ducting section. The at least one source of ultraviolet radiation may be recessed from the one or more walls. Recessing the at least one source of ultraviolet radiation mitigates obstruction of the airflow within the air handling system.

The air cleaning system may further comprise a reflective surface arranged to reflect ultraviolet radiation emitted by the source of ultraviolet radiation within the interior volume of the ducting section. The use of a reflective surface allows photons emitted by the source of ultraviolet radiation to be reflected back into the interior volume of the ducting section, which increases the energy flux density within the interior volume. The reflective surface may comprise a material that is capable of reflecting at least 65% of incident ultraviolet radiation. Preferably, the reflective surface may comprise a material that is capable of reflecting at least 70% of incident ultraviolet radiation. More preferably, the reflective surface may comprise a material that is capable of reflecting at least 75% of incident ultraviolet radiation. More preferably, the reflective surface may comprise a material that is capable of reflecting at least 80% of incident ultraviolet radiation. More preferably, the reflective surface may comprise a material that is capable of reflecting at least 85% of incident ultraviolet radiation. More preferably, the reflective surface may comprise a material that is capable of reflecting at least 90% of incident ultraviolet radiation. More preferably, the reflective surface may comprise a material that is capable of reflecting at least 95% of incident ultraviolet radiation. Reflecting an increased amount of incident ultraviolet radiation further increases the energy flux density within the interior volume.

The material may comprise one or more of: polytetrafluoroethylene, PTFE, nylon, ultra-high-molecular-weight polyethylene, UHMWPE, or any combination of the foregoing materials. Preferably, the material comprises PTFE, which has high reflectivity to ultraviolet radiation.

The air cleaning system may further comprise a removable casing, wherein the source of ultraviolet radiation is disposed within the removable casing. Providing the source of ultraviolet radiation within a removable casing allows the air cleaning system to be maintained. Specifically, providing the source of ultraviolet radiation within a removable casing allows the source of ultraviolet radiation (e.g. one or more ultraviolet lamps) to be replaced when necessary.

The removable casing may further comprise the reflective surface. The removable casing may comprise a back wall and side walls that define an enclosure in which the source of ultraviolet radiation is disposed. The reflective surface may be disposed on the back wall of the removable casing.

The ducting section may comprise at least one wall that defines the interior volume, wherein the removable casing is arranged to cover an opening in the at least one wall. The ducting section may comprise a plurality of walls that define the interior volume, wherein the air cleaning system comprises a plurality of removable casings, each of the plurality of removable casings being arranged to cover an opening in a respective one of the plurality of walls.

Each of the plurality of removable casings may comprise a source of ultraviolet radiation. By providing a source of ultraviolet radiation in each of a plurality of removable casings, the airflow through the internal volume is exposed to ultraviolet radiation from multiple directions, which increases the irradiation that each virus is exposed to.

The at least one source of ultraviolet radiation may comprise a plurality of ultraviolet lamps. The ultraviolet lamps may be mercury lamps. The ultraviolet lamps may be amalgam lamps. The ultraviolet lamps may be LEDs. The at least one source of ultraviolet radiation may comprise an excimer lamp or excimer plate.

Adjacent ones of the plurality of ultraviolet lamps may be spaced apart from one another to provide a gap between the adjacent ones of the plurality of ultraviolet lamps. A portion of the reflective surface may be exposed to the ultraviolet radiation through the gap between the adjacent ones of the plurality of ultraviolet lamps. Providing a gap between adjacent lamps allows a portion of the reflective surface to be exposed to the ultraviolet radiation. This means that a greater portion of the ultraviolet radiation is reflected, thereby increasing the energy flux density within the interior volume.

The total area of the gaps may be between about 50% and about 80% of an area of a surface on which the ultraviolet lamps are disposed. The total area of the gaps may be between about 70% and about 80% of the area of the surface on which the ultraviolet lamps are disposed. The total area of the gaps may be between about 75% and about 80% of the area of the surface on which the ultraviolet lamps are disposed. Increasing the total area of the gaps increases the amount of reflected ultraviolet radiation and therefore maximises the energy flux density within the interior volume.

Each of the plurality of ultraviolet lamps may comprise a longitudinal axis, wherein the longitudinal axis of each of the plurality of lamps is parallel to a flow path from the inlet to the outlet. This maximises the time that each virus particle is exposed to the ultraviolet radiation from the lamps.

The ducting section may comprise a plurality of walls that define, in part, the interior volume. The at least one source of ultraviolet radiation may comprise a first source of ultraviolet radiation configured to emit ultraviolet radiation into the interior volume from a first wall of the plurality of walls, and a second source of ultraviolet radiation configured to emit ultraviolet radiation into the interior volume from a second wall of the plurality of walls. The second wall may be nonparallel to the first wall. The second wall may be orthogonal to the first wall. This configuration of the ultraviolet lamps means that the airflow through the internal volume is exposed to ultraviolet radiation from multiple directions, which increases the irradiation that each virus is exposed to and reduces the likelihood of radiation being blocked by larger particles.

The at least one source of ultraviolet radiation may be arranged to emit ultraviolet-C, UVC, radiation. The wavelength of the UVC radiation may be between about 200 nm and about 280 nm. Preferably, the wavelength of the UVC radiation is between 210 nm and 260 nm. More preferably, the wavelength of the UVC radiation is about 222 nm or about 254 nm. 222 nm and 254 nm radiation have been shown to be effective at killing pathogens. In addition, 254 nm ultraviolet lamps are widely available, therefore providing a cost-effective solution.

According to a third aspect of the present disclosure, there is provided an air handling system comprising: at least one source of ultraviolet radiation; and a device according to the first aspect. The air handling system may further comprise an air cleaning system according to the second aspect. The air cleaning system may comprise the source of ultraviolet radiation. The air handling system may be a heating, ventilation and air conditioning, HVAC, system.

The device may be a first device located upstream of the at least one source of ultraviolet radiation. The air handling system may further comprise a second device according to the first aspect, wherein the second device is located downstream of the at least one source of ultraviolet radiation. Implementing two devices provides increased reflection of ultraviolet radiation, thereby providing increased energy flux density in the region of the at least one source of ultraviolet radiation, and consequently further reducing the electrical power needed to provide an energy flux density using the at least one source of ultraviolet radiation.

The device may be a radiation-reflecting device. The air handling system may further comprise a radiation-attenuating device for attenuating ultraviolet radiation. The radiation-reflecting device may be located between the at least one source of ultraviolet radiation and the radiation-attenuating device. Implementing separate radiation-reflecting and radiation-attenuating devices means that the radiation-reflecting device and radiation-attenuating device can have different elongate flow passage diameters.

The radiation-reflecting device may be a first radiation-reflecting device located upstream of the at least one source of ultraviolet radiation. The air handling system may further comprise a second device according to the first aspect. The second device may be a second radiation-reflecting device and may be located downstream of the at least one source of ultraviolet radiation. The air handling system may further comprise a second radiation-attenuating device for attenuating ultraviolet radiation. The second radiation-reflecting device may be located between the at least one source of ultraviolet radiation and the second radiation-attenuating device.

The radiation-attenuating device may comprise a plurality of elongate flow passages. Each of the elongate flow passages may be configured to permit airflow through the elongate flow passage.

Each of the elongate flow passages may comprise one or more internal walls comprising a coating configured to absorb a proportion of incident ultraviolet radiation. The radiation-attenuating device may be arranged for insertion in a ducting section of the air handling system. The radiation-attenuating device may be arranged for insertion in the ducting section such that any airflow through the ducting section flows through the elongate flow passages of the radiation-attenuating device. The plurality of elongate flow passages may be arranged such that, collectively, the plurality of elongate flow passages has a substantially square or rectangular cross-section. Each of the plurality of elongate flow passages may be configured to permit air to flow substantially unimpeded through the elongate flow passage from the inlet to the outlet. Each of the plurality of elongate flow passages may be straight. Each of the plurality of elongate flow passages may have an aspect ratio calculated by dividing a length of the elongate flow passage by a diameter of the elongate flow passage. The aspect ratio of each of the plurality of elongate flow passages may be greater than or equal to 4. The aspect ratio of each of the plurality of elongate flow passages may be less than or equal to 50, optionally less than or equal to 20. The coating may be configured to absorb a proportion of incident UVC radiation. The coating may be configured to reflect less than 60% of incident UVC radiation, optionally less than 20% of incident UVC radiation. The coating may comprise one or more coats of black paint.

According to a fourth aspect, there is provided a method, comprising: installing a device according to the first aspect in an air handling system comprising a source of ultraviolet radiation. The method may further comprise installing an air cleaning system according to the second aspect in the air handling system. The air cleaning system may comprise the source of ultraviolet radiation.

According to a fifth aspect, there is provided an air handling system, comprising: at least one source of ultraviolet radiation; one or more radiation-attenuating devices for attenuating ultraviolet radiation; and a reflective surface configured to reflect incident ultraviolet radiation away from the one or more radiation-attenuating devices.

The use of the reflective surface increases the energy flux density in the region of the ultraviolet radiation source, as a result of reflecting incident ultraviolet radiation away from the one or more radiation-attenuating devices (i.e. back towards the ultraviolet radiation source). The reflection of ultraviolet radiation back towards the source of ultraviolet radiation means that the power requirement to provide a given energy flux density in the vicinity of the source of ultraviolet radiation is reduced. The given energy flux density may be the energy flux density required to provide a desired level of inactivation of one or more types of pathogen within the airflow.

The reflective surface may be disposed between the at least one source of ultraviolet radiation and the one or more radiation-attenuating devices. The reflective surface may be provided on a face of the one or more radiation-attenuating devices. As used herein, the phrase “between the at least one source of ultraviolet radiation and the one or more radiation-attenuating devices” is intended to encompass providing the reflective surface on a face of the one or more radiation-attenuating devices that is closest to the at least one source of ultraviolet radiation.

The air handling system may further comprise a baffle disposed between the at least one source of ultraviolet radiation and the one or more radiation-attenuating devices, and wherein the reflective surface is provided on a face of the baffle. Providing the reflective surface on a face of a baffle means that ultraviolet radiation can be reflected away from the one or more radiation-attenuating devices without necessarily requiring a retroreflective surface.

The system may comprise a plurality of baffles disposed between the at least one source of ultraviolet radiation and the one or more radiation-attenuating devices, wherein each of the plurality of baffles includes a reflective surface configured to reflect incident ultraviolet radiation away from the one or more radiation-attenuating devices. Providing a plurality of baffles reduces the likelihood of ultraviolet photons reaching the one or more radiation-attenuating devices without first being reflected by a reflective surface. In particular, a plurality of baffles can be provided to ensure that any ultraviolet photos travelling orthogonal to a cross-section of the air handling system are incident on one or more of the plurality of reflective surfaces.

The system may comprise a plurality of reflective surfaces. This reduces the likelihood of ultraviolet photons reaching the one or more radiation-attenuating devices without first being reflected by a reflective surface. The plurality of reflective surfaces may be arranged such that any ultraviolet photos travelling orthogonal to a cross-section of the air handling system are incident on one or more of the plurality of reflective surfaces. Such an arrangement further reduces the likelihood of ultraviolet photons reaching the one or more radiation-attenuating devices without first being reflected by a reflective surface.

The plurality of reflective surfaces may be arranged such that one or more of the plurality of reflective surfaces prevent airflow from bypassing the one or more radiation-attenuating devices. This means that ultraviolet photons are also prevented from bypassing the one or more radiation-attenuating devices, increasing attenuation of the radiation.

The reflective surface may be retroreflective to incident ultraviolet radiation. The reflective surface may be configured to reflect incident UVC radiation away from the one or more radiation-attenuating devices. The air handling system may be a heating, ventilation and air conditioning (HVAC) system.

Each of the one or more radiation-attenuating devices may comprise a plurality of elongate flow passages, wherein each of the elongate flow passages is configured to permit airflow through the elongate flow passage. The one or more radiation-attenuating devices may be arranged such that any airflow through the air handling system flows through one of the elongate flow passages of one of the one or more radiation-attenuating devices.

The one or more radiation-attenuating devices may be arranged such that a direction of airflow through the elongate flow passages is nonparallel to a direction of airflow past the at least one source of ultraviolet radiation. The one or more radiation-attenuating devices may be arranged such that the direction of airflow through the elongate flow passages is substantially perpendicular to the direction of airflow past the at least one source of ultraviolet radiation. Arranging the one or more radiation-attenuating devices nonparallel to the direction of airflow reduces the likelihood of reflected ultraviolet radiation passing through the one or more radiation-attenuating devices without being attenuated by the one or more radiation-attenuating devices.

Each of the plurality of elongate flow passages may comprise one or more internal walls configured to absorb a proportion of incident ultraviolet radiation. The one or more internal walls of each elongate flow passage may comprise a coating configured to absorb a proportion of incident ultraviolet radiation. The coating may be configured to reflect less than 60% of incident UVC radiation. The coating may be configured to reflect less than 20% of incident UVC radiation. The coating may comprise one or more coats of black paint.

The air handling system may comprise an air cleaning system according to the second aspect. To further increase the energy flux density in the region of the ultraviolet radiation source, the air handling system may include reflective surfaces disposed upstream of the ultraviolet radiation source and downstream of the one or more ultraviolet radiation sources. One or more radiation-attenuating devices may also be provided both upstream and downstream of the ultraviolet radiation source, with the reflective surfaces being provided between the ultraviolet radiation source and a corresponding one or more radiation-attenuating devices.

### BRIEF DESCRIPTION OF FIGURES

Specific embodiments are described below by way of example only and with reference to the accompanying drawings, in which:

FIG. 1 is a schematic diagram of a first air handling system comprising a device for reflecting ultraviolet radiation.

FIG. 2 is a front view of a device for reflecting ultraviolet radiation disposed within a ducting section.

FIG. 3A is a cross-section through line A-A in FIG. 2.

FIG. 3B is a perspective view of a single elongate flow passage of the device shown in FIG. 2, in isolation from the other elongate flow passages of the device.

FIG. 4A is a schematic diagram indicating how ultraviolet radiation is reflected by specular reflection in an elongate flow passage.

FIG. 4B is a schematic diagram indicating how ultraviolet radiation is reflected by diffuse reflection in an elongate flow passage.

FIG. 4C is a schematic diagram indicating how ultraviolet radiation is reflected by retroreflection in an elongate flow passage.

FIG. 5 is a schematic diagram of a ducting section comprising a device for reflecting ultraviolet radiation.

FIG. 6 is a top perspective view of a ducting section of an air cleaning system.

FIG. 7 is a side view of the ducting section of FIG. 6.

FIG. 8 is a top perspective view of a casing arranged for attachment to the ducting section of FIG. 6.

FIG. 9 is a bottom perspective view of the casing of FIG. 8.

FIG. 10 is a schematic diagram of the arrangement of lamps within a casing.

FIG. 11 is a schematic diagram of a bevelled corner member of a ducting section.

FIG. 12 is a top perspective view of an air cleaning system in which the casing of FIG. 8 is attached to the ducting section of FIG. 6.

FIG. 13 is a side view of the air cleaning system of FIG. 12.

FIG. 14 is a cross-sectional view through line A-A in FIG. 13.

FIG. 15 is a nomograph providing parameter values for surface decontamination.

FIG. 16 is a schematic diagram of photons impinging on particles within a volume.

FIG. 17 is a nomograph providing parameter values for decontamination of a volume of air.

FIG. 18 is a schematic diagram illustrating reflection of a collimated beam between two surfaces.

FIG. 19 is a schematic diagram illustrating reflection of a beam within a spherical cavity.

FIG. 20 is a schematic diagram of a second air handling system comprising two devices for reflecting ultraviolet radiation.

FIG. 21 is a schematic diagram of a third air handling system.

FIG. 22 is a schematic diagram of a fourth air handling system.

FIG. 23 is a schematic diagram of a fifth air handling system.

FIG. 24 is a schematic diagram of an air handling system implemented in a HVAC system of a building.

### DETAILED DESCRIPTION

Implementations of the present disclosure are explained below with particular reference to reflection of UVC photons within an air handling system that comprises an air cleaning system (which may also be referred to as an air sanitising system) utilising UVC radiation to inactivate pathogens in airflow through the air cleaning system. It will be appreciated, however, that the devices, systems and methods described herein are also applicable to reflecting UVC photons in other systems, in which it is desirable to increase the flux density of UVC photons in a first location and reduce leakage of UVC photons to a second location. It will further be appreciated that the implementations described herein are not limited to reflection of UVC photons, but may also be used to reflect photons over the entire electromagnetic spectrum.

In general terms, a device as disclosed herein can be disposed between a first location at which a source of electromagnetic radiation (e.g. ultraviolet radiation) is used to provide a particular flux density, and a second location. Disposing the device between the first location and the second location results in an increase in flux density at the first location, while permitting fluid flow between the first location and the second location. This is achieved by reflecting photons incident on a reflective surface of the device back towards the first location.

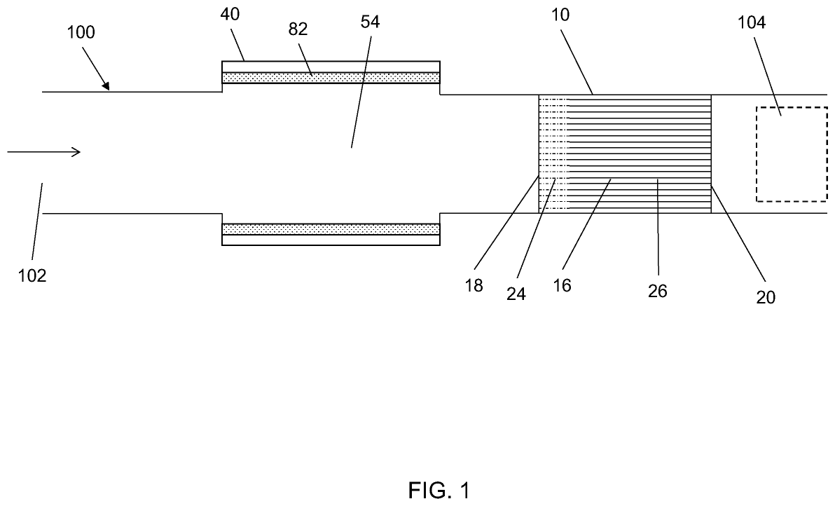

An example air handling system 100 that comprises a device 10 for reflecting UV radiation is shown schematically in FIG. 1. In one example, the air handling system 100 is a heating, ventilation, and air conditioning (HVAC) system, although other air handling systems are contemplated in the present disclosure. The air handling system 100 comprises an inlet 102. The inlet 102 may receive a proportion of air that is recirculated from within a building in which the air handling system 100 is implemented. The direction of airflow is indicated by the arrow in FIG. 1.

At least one ultraviolet radiation source (shown in FIG. 1 as ultraviolet lamps 82) is located downstream of the inlet 102. In the example shown in FIG. 1, the ultraviolet lamps 82 are housed in an air cleaning system 40 that forms part of the air handling system 100. As explained further below, the ultraviolet lamps 82 are recessed from the interior volume 54 of the air cleaning system 40, in order to mitigate obstruction of airflow within the air cleaning system 40. The ultraviolet lamps 82 provide an energy flux density within an interior volume of the air cleaning system 40.

In the example shown in FIG. 1, the device 10 for reflecting UV radiation is located downstream of the air cleaning system 40. Specifically, the device 10 is located between the air cleaning system 40 and a particular location 104 within the air handling system 100. As described in more detail below, the device 10 includes a plurality of elongate flow passages 16, each of which permits air to flow through the elongate flow passage 16 from the interior volume 54 of the air cleaning system 40 to the particular location 104.

The device 10 also includes a first end 18 and a second end 20. Each elongate flow passage comprises two flow passage portions: a first flow passage portion 24 (indicated by the dash-dot lines of the elongate flow passages 16 in FIG. 1), which is adjacent to the first end 18, and a second flow passage portion 26 (indicated by the solid lines of the elongate flow passages 16 in FIG. 1), which is adjacent to the second end 20. In the configuration shown in FIG. 1, the first end 18 of the device 10 is located closer to the ultraviolet lamps 82 of the air cleaning system 40 than the second end of the device 20. In other words, the distance between the first end 18 of the device 10 and the ultraviolet lamps 82 is less than the distance between the second end 20 of the device 10 and the ultraviolet lamps 82.

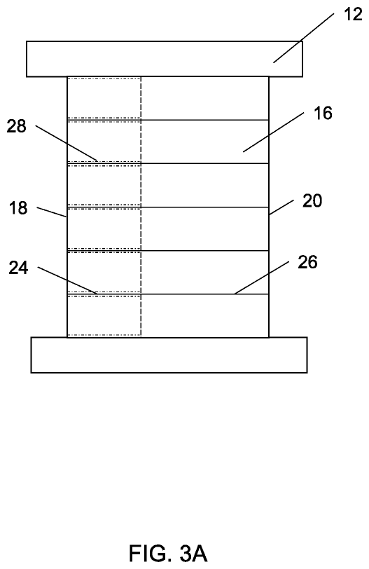

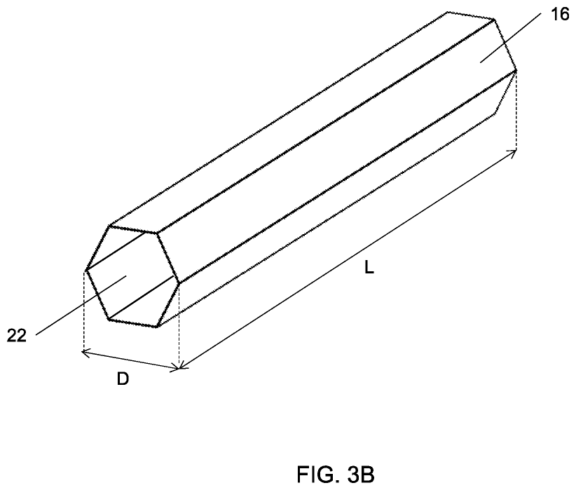

The device 10 includes one or more reflective surfaces (shown in FIG. 3A) configured to reflect incident ultraviolet radiation. The one or more reflective surfaces are provided on the internal walls of the first flow passage portion 24 of each elongate flow passage 16. As described further below, the one or more reflective surfaces of the device 10 are configured to reflect ultraviolet radiation back towards the interior volume 54 of the air cleaning system 40, which results in increased energy flux density within the interior volume 54 of the air cleaning system 40.

The increase in energy flux density arising from the one or more reflective surfaces of the device 10 means that the total electrical power required in order to generate the required energy flux density within the interior volume 54 is reduced, compared to an air handling system in which the device 10 is not implemented. This means that fewer ultraviolet lamps 82 and/or lower power ultraviolet lamps 82 may be utilised to achieve the required energy flux density within the interior volume 54, resulting in increased energy efficiency for inactivation of airborne pathogens.

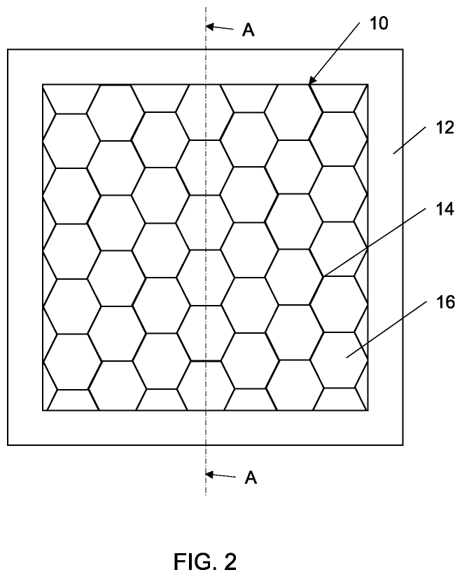

The device 10 implemented in the air handling system 100 shown in FIG. 1 will now be described with reference to FIGS. 2 to 5. The air cleaning system 40 implemented in the air handling system 100 shown in FIG. 1 will then be described with reference to FIGS. 6 to 19. As one example, the air cleaning system 40 may be provided in the form of the air cleaning device described in UK patent application no. 2018738.1, the contents of which are hereby incorporated by reference.

FIG. 2 is a front view of the device 10, while FIG. 3A shows a cross-section through the device 10. In the example shown in FIGS. 2 and 3A, the device 10 is disposed within a ducting section having a square cross-section, which is indicated schematically at 12. The device 10 may be installed within an existing ducting section of an air handling system. Alternatively, the device 10 may be provided as part of a ducting section for an air handling system. In the latter case, the device 10 may be installed in the air handling system by replacing an existing ducting section of the air handling system with ducting section that comprises the device 10.

The device 10 comprises a honeycomb structure 14 that defines a plurality of elongate flow passages 16. As shown in FIGS. 1 and 3A, the individual elongate flow passages 16 have a hexagonal cross-section. The honeycomb structure 14 allows air to pass through the device 10 (i.e. through the elongate flow passages 16). The honeycomb structure 14 helps to damp out turbulence in the airflow, which can provide beneficial effects downstream, such as reduced mixing and reduced frictional pressure drop. The honeycomb structure 14 may, for example, be a Hexweb® aluminium honeycomb available from Hexcel Corporation of Stamford, CT, USA. Such honeycomb is available with wall thicknesses of between 0.0007 inches (0.3 mm) and 0.004 inches (1.6 mm).

The honeycomb structure 14 is formed of a material that is not degraded by ultraviolet radiation, so that the device 10 is not damaged by exposure to an ultraviolet radiation source. For example, the honeycomb structure 14 may be formed of aluminium.

The honeycomb structure 14 has a substantially square or rectangular cross-section, allowing it to be disposed within a square or rectangular cross-section of a ducting section (e.g. the square cross-section of the ducting section 12 shown in FIG. 1). This means that, collectively, the plurality of elongate flow passages 16 has a substantially square or rectangular cross-section. The edges of the honeycomb structure 14 will, of course, be non-straight, owing to the tessellation of the hexagonal elongate flow passages 16. Accordingly, the cross-section of the honeycomb structure 14 will not be a strict square or rectangle shape. In this context, therefore, “substantially square or rectangular” means that the overall envelope defined by the corners of the honeycomb structure 14 is square or rectangular, without requiring the edges of the honeycomb structure 14 to be straight.

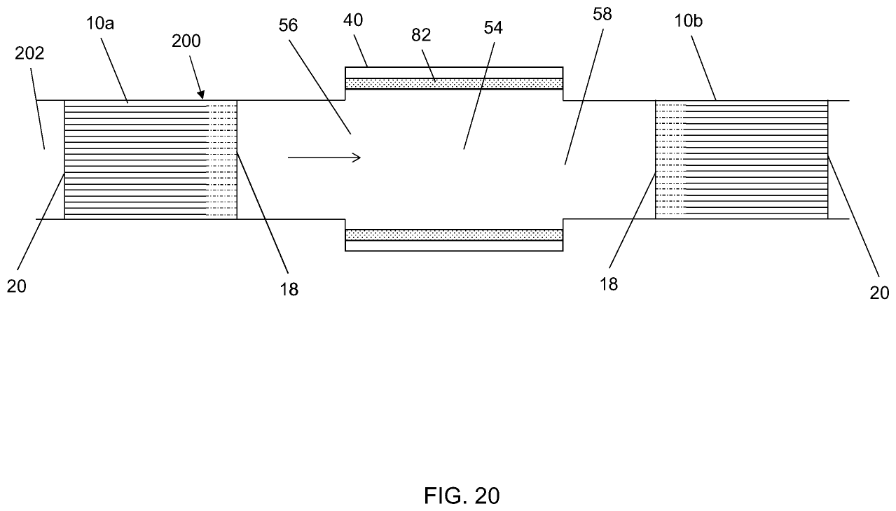

As shown in FIG. 3A, the device 10 (specifically, the honeycomb structure 14) comprises a first end 18 and a second end 20. In the configuration shown in FIG. 1, the first end 18 defines an inlet to the device 10, while the second end 20 defines an outlet from the device 10. In other configurations, however, the second end 20 may define the inlet to the device 10 and the first end 18 may define the outlet from the device 10 (e.g. as with the first device 10a in the air handling system 200 shown in FIG. 20). More specifically, the first end 18 defines a first end of each of the elongate flow passages 16, while the second end 20 defines a second end of each of the elongate flow passages 16. Each elongate flow passage 16 of the honeycomb structure 14 allows air to flow between the first end 18 and the second end 20 of the device 10 (either from the first end 18 to the second end 20 or from the second end 20 to the first end 18). In particular, the honeycomb structure 14 fills an internal volume defined by the ducting section 12, such that any air flowing through the ducting section 12 passes through an elongate flow passage 16.

Each elongate flow passage 16 comprises one or more internal walls 22 (indicated in FIG. 3B). Specifically, in the example of an elongate flow passage 16 with a hexagonal cross-section (i.e. as shown in FIG. 2), each elongate flow passage 16 has six internal walls 22. As indicated by the vertical dashed line in FIG. 3A, each elongate flow passage 16 comprises a first flow passage portion 24 and a second flow passage portion 26. The first flow passage portion 24 is adjacent to the first end 18 of the device 10, while the second flow passage portion 26 is adjacent to the second end 20 of the device 10. Each first flow passage portion 24 extends along approximately 10% to 40% of the total length of its respective elongate flow passage 16, preferably between 20% and 30%, and more preferably about 25% of the total length of its respective elongate flow passage 16.

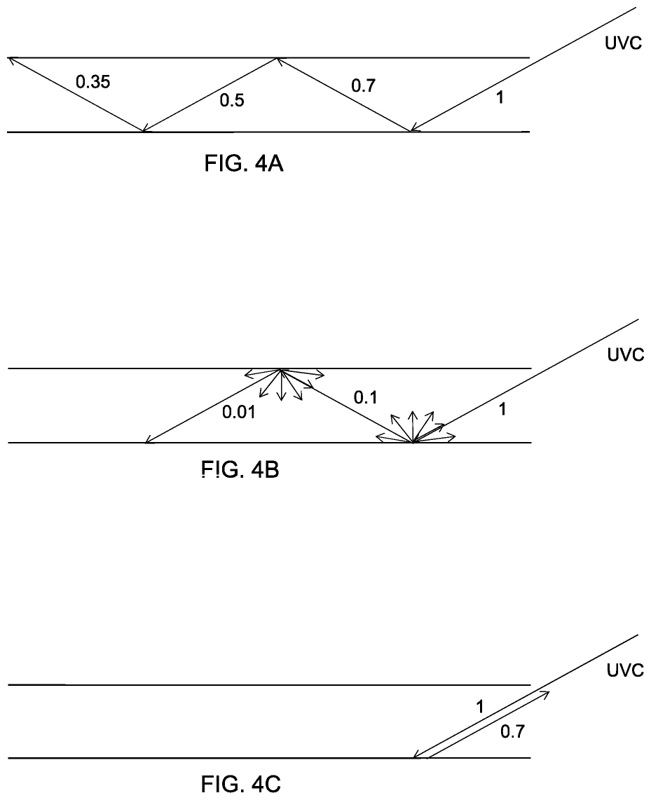

In the first flow passage portion 24 of each elongate flow passage 16, the internal walls 22 have a retroreflective surface 28 (indicated by the dash-dot lines in FIG. 3A). The retroreflective surface 28 of each elongate flow passage 16 is configured to reflect UVC radiation entering the first end 18 of the device 10 by retroreflection. This means that the retroreflective surface 28 of each elongate flow passage 16 reflects UVC radiation away from the second end 20 of the device 10. Specifically, UVC rays that are incident on the retroreflective surface 28 at a particular angle of incidence are reflected back on themselves at an angle that is substantially equal to the angle of incidence, as shown schematically in FIG. 4C. The above values relating to the length of each flow passage portion 24 are preferable because retroreflective surfaces are more effective at reflecting incident radiation when the angle of incidence is close to normal.

Preferred lengths of each first flow passage portion 24 may also be expressed in terms of the diameter of its respective elongate flow passage 16 (indicated as “D” in FIG. 3B). Specifically, the length of each first flow passage portion 24 may be between one and three times the diameter of its respective elongate flow passage 16. Preferably, the length of each first flow passage portion 24 may be between two and three times the diameter of its respective elongate flow passage 16. More preferably, the length of each first flow passage portion 24 may be about two times the diameter of its respective elongate flow passage 16.

In one example, the retroreflective surface 28 is provided in the form of a retroreflective coating on the internal walls 22 of the first flow passage portion 24 of each elongate flow passage 16. As one particular example, the retroreflective coating comprises Scotchlite® retroreflective material available from 3M of Maplewood, Minnesota, U.S., which is made by embedding glass microspheres that have an aluminium reflective coating on half of the sphere in a binder material.

In alternative examples, the retroreflective coating comprises microspheres made from quartz and/or fused silica that transmits at 254 nm. An aluminium reflective coating is provided on half of each sphere. Aluminium has a reflectivity of over 70% for UVC radiation. Forming the microspheres of quartz and/or fused silica is expected to improve the transmissibility of ultraviolet radiation through each microsphere to the aluminium surface. The retroreflective coating may further be provided in the form of a paint that includes a binder such as silicone, which transmits UVC radiation.

As one specific example manufacturing method, fused silica microspheres may be initially coated with an aluminium coating on one side (i.e. to form a hemispherical coating). The aluminium coating offsets the centre of gravity of each microsphere, which for an uncoated microsphere is at the centre of the microsphere. The coated microspheres may then be placed into a liquid binder material or glue having a viscosity that is sufficiently low as to allow the microspheres to orient in the liquid such that the aluminium coating is oriented downwards. The coated microspheres orient themselves in the liquid as a result of the offset centre of gravity provided by the aluminium coating. Examples of liquid binder materials or glues that have sufficiently low viscosity to allow the coated microspheres to orient under gravity include silicones, acrylics and epoxies, among others. The choice of particular material will depend on its effective viscosity, bond strength and UV compatibility. In this context, UV compatibility refers to the optical properties of the material in the UVC spectrum (i.e. the ability to transmit incident UVC radiation to the microspheres), as well as the ability of the material to withstand irradiation by UVC photons while maintaining its structural integrity. As one specific example, cyanoacrylates such as ethyl 2-cyanoacrylate may be used as a suitable glue.

As an alternative to a retroreflective coating, the material of the honeycomb structure 14 may be embossed, in the first flow passage portions 24, with a plurality of corner cube reflectors, which act to reflect incident radiation by retroreflection. In this way, the retroreflective surface 28 may be provided using the aluminium material of the honeycomb structure 14.

As one example, the retroreflective surface 28 may be applied to a first flow passage portion 24 of each elongate flow passage of the device for attenuating ultraviolet radiation described in UK patent application no. 2118916.2, the contents of which are hereby incorporated by reference.

Methods of manufacturing the elongate flow passages 16 will now be described. In one example, a honeycomb structure 14 is provided. One end of the honeycomb structure 14 is then dipped in an adhesive such as ethyl 2-cyanoacrylate. The depth of the honeycomb structure 14 that is dipped in the adhesive is equivalent to the desired length of the first elongate flow passage portion 24 (e.g. 25% of the length of the honeycomb structure). The honeycomb structure 14 is then removed from the adhesive. The portion of the honeycomb structure 14 that was dipped in the adhesive is then dipped in a container comprising a plurality of microspheres, such that the microspheres adhere to the adhesive. The microspheres may be oriented in a particular direction before the honeycomb structure 14 is dipped in the microspheres. For example, the offset centre of gravity of the microspheres can be exploited to orient the microspheres using an external force such as gravity, centrifugal force or electrostatic force. Variations in surface tension could also be exploited. Alternatively, the microspheres may be randomly oriented in the container, such that the microspheres have a random orientation on the retroreflective surface 28. In this case, the microspheres are still expected to provide a substantial retroreflection effect.

In an alternative example, the retroreflective surface 28 may be applied to sheets of aluminium that are subsequently formed into the honeycomb structure 14. For example, the retroreflective surface 28 may be coated on each side of an end portion of a plurality of aluminium sheets. In particular, a band of the retroreflective surface 28 is applied parallel to an end of each aluminium sheet. Multiple lines of adhesive may then be applied to each sheet, perpendicular to the end of the sheet. The adhesive lines applied to each subsequent sheet are offset from the adhesive lines applied to the previous sheet. This means that, when the top and bottom sheets are pulled apart from each other in opposing directions normal to the planes of the sheets, a honeycomb structure 14 is formed.

As a further alternative example, the honeycomb structure 14 may be formed of interlocking horizontal and vertical “comb-like” sheets. In such an example, each of the horizontal sheets includes a series of slots extending a certain distance through the sheet (e.g. 50% of the distance). Likewise, each of the vertical sheets includes a series of slots extending a corresponding distance (e.g. 50%) through the sheet, such that the combined length of a slot in a horizontal sheet and a slot in a vertical sheet is at least the length of the sheet. The horizontal and vertical sheets are then slotted together, to form a honeycomb structure 14 with a quadrilateral (e.g. square) cross-section. This method of manufacturing a honeycomb structure 14 is particularly suitable where the retroreflective surface 28 is provided in the form of embossed corner cube reflectors in each sheet.

As shown in FIG. 3A, each elongate flow passage 16 is straight. This minimises the resistance to air flowing through the elongate flow passage 16 from the first end 18 to the second end 20. In turn, this minimises the pressure drop within the air handling system as a result of implementing the device 10. Each elongate flow passage 16 is also configured to permit air to flow substantially unimpeded through the elongate flow passage 16 between the first end 18 and the second end 20. Each elongate flow passage 16 will, of course, cause some impediment to air flow through the elongate flow passage 16 (as a result of the boundary layer at the surface of the internal walls 22 of the elongate flow passage 16). In this context, therefore, “substantially unimpeded” means that nothing protrudes from the internal walls 22 towards the centre of the elongate flow passage 16, and the internal walls 22 themselves are not curved or angled in the direction of airflow, while recognising that boundary layer effects will be present within the elongate flow passage 16.

FIG. 3B shows an individual elongate flow passage 16 of the device 10 shown in FIGS. 1 and 2. The hexagonal cross-section of the elongate flow passage 16 can be seen in FIG. 3B. As shown schematically in FIG. 3B, the length of each elongate flow passage 16 is significantly greater than the diameter of the elongate flow passage 16. In this context, “significantly greater” is to be interpreted as four or more times greater.

In the second flow passage portion 26 of the elongate flow passage 16 (shown in FIG. 3A), the internal walls 22 of the elongate flow passage 16 are configured to attenuate ultraviolet radiation that is incident on the internal walls 22.

A desired level of attenuation of the ultraviolet radiation can be achieved by tuning a number of parameters of the elongate flow passages 16. For example, the desired level of attenuation may be to below the RG2 (Risk Group 2) actinic UV limit defined in standards IEC 62471:2006 and BS EN 62471:2008 (Photobiological safety of lamps and lamp systems). The RG2 actinic UV limit is 0.03 W/m². Specifically, a desired level of attenuation can be achieved by varying one or more of: (i) an aspect ratio of the elongate flow passage 16; (ii) the nature of the reflection of incident radiation from the internal walls 22 of the second flow passage portions 26; and (iii) the reflectivity of the internal walls 22 of the second flow passage portions 26 to ultraviolet radiation. The effect of varying these parameters is explained in the following paragraphs.

The aspect ratio of an elongate flow passage 16 is defined as the ratio of the length (L in FIG. 3B) of the elongate flow passage 16 to the diameter (D in FIG. 3B) of the elongate flow passage 16. In other words, the aspect ratio may be calculated by dividing the length of the elongate flow passage 16 by the diameter of the elongate flow passage 16.

Although the streamlines of air flowing in the device 10 are parallel to the internal walls 22, optical radiation enters the honeycomb structure 14 at a variety of angles. Radiation that enters the honeycomb structure 14 normally (i.e. parallel to the elongate flow passages 16) will pass through relatively unimpeded. Radiation that enters the honeycomb structure 14 at an oblique angle above a certain threshold determined by the length of the first flow passage portion 24 will be reflected back on itself by the retroreflective surface 28. For radiation that enters the honeycomb structure 14 at oblique angles above a certain threshold determined by the aspect ratio but below the threshold at which photons collide with the retroreflective surface 28, the photons will collide with the internal walls 22 of the second flow passage portion 26 at least once.

For photons that collide with the internal walls 22 of the second flow passage portion 26 of the elongate flow passage 16 at a first aspect ratio (e.g. 4:1), increasing the aspect ratio (e.g. to 10:1) increases the number of reflections of the photon within the second flow passage portion 26 of the elongate flow passage 16. In addition, increasing the aspect ratio of the elongate flow passage 16 reduces the range of angles over which a photon can pass through the elongate flow passage 16 without colliding with the internal walls 22 of the second flow passage portion 26.

The number of reflections of a photon increases with the obliqueness of the incident angle. For example, an elongate flow passage 16 with an aspect ratio of 10:1 will allow photon rays at incident angles of approximately +/−5.7 degrees from normal to pass through the elongate flow passage 16 without colliding with the internal walls 22. Rays at incident angles from 5.7 degrees to about 16 degrees from normal will pass through the elongate flow passage 16 with a single reflection off the internal walls 22. Rays at incident angles from about 16 degrees to about 29 degrees will pass through the elongate flow passage 16 with two reflections off the internal walls 22.

The above discussion is applicable to the case of specular reflection. When radiation is reflected by specular reflection, a ray is reflected at an equal but opposite angle to the angle of incidence, as shown in FIG. 4A. For example, if a ray of ultraviolet photons collides with an internal wall 22 at an incidence angle of 20 degrees to the internal wall 22, measured between the incident ray and the internal wall 22 before the collision point, then the ray is reflected at a reflected angle of 20 degrees to the internal wall 22 in the opposite direction (that is, measured between the reflected ray and the internal wall 22 after the collision point). Specular reflection would occur, for example, if the internal walls 22 were bare aluminium. For UVC radiation, the reflectivity of bare aluminium is approximately 70%, meaning that rays with two collisions would be attenuated by about half, as indicated by the magnitudes in FIG. 4A. The level of attenuation would be higher for oblique rays greater than 29 degrees.

It will be clear from the above discussion that increasing the aspect ratio increases the level of attenuation of ultraviolet radiation. However, very high aspect ratios would create large boundary layer effects through each of the elongate flow passages 16, resulting in a pressure drop. For this reason, the aspect ratio is preferably less than or equal to 50:1. More preferably, the aspect ratio is less than or equal to 20:1, in order to further reduce the pressure drop resulting from usage of the device 10.

A further downside to high aspect ratios is that the device 10 would occupy a large amount of space within the air handling system. An aspect ratio of less than or equal to 50:1 is also preferred in order to minimise the volume taken up by the device 10, and an aspect ratio of less than or equal to 20:1 is more preferred in order to further minimise the volume of the device 10.

One way of minimising the volume of the device 10 would be to reduce the absolute diameter of the elongate flow passages 16. For example, halving the diameter of an elongate flow passage 16 means that the length of an elongate flow passage 16 can also be halved, in order to achieve a given aspect ratio.

However, using smaller diameter elongate flow passages 16 means that the device 10 comprises a greater number of elongate flow passages 16. In other words, more elongate flow passages 16 are required in order to fill the cross-sectional area of the ducting section 12. The internal walls 22 have a given thickness (e.g. between 0.3 mm and 1.6 mm for a honeycomb structure 14 formed from Hexweb® aluminium honeycomb). Therefore, increasing the number of elongate flow passages 16 increases the proportion of the cross-section of the device 10 that is occupied by the honeycomb structure 14 itself (and correspondingly reduces the open area of the device 10 provided by the elongate flow passages).

Reducing the open area of the device 10 impedes air flow through the device 10, thereby increasing the pressure drop through the device 10. It is therefore preferable for the elongate flow passages 16 to have a diameter of between 6 mm and 12 mm. This range also corresponds to the ranges of honeycomb cell sizes of honeycomb structures formed from Hexweb® aluminium honeycomb.

The above discussion regarding increasing the number of collisions by increasing the aspect ratio assumes that the reflection off the internal walls 22 of the second flow passage portion 26 of each elongate flow passage 16 is specular reflection (as shown in FIG. 4A). The number of collisions within the second flow passage portion 26 of an elongate flow passage 16 can be increased by making the reflection off the internal walls 22 of the more diffuse (i.e. where the incident radiation is scattered in a range of directions, as shown in FIG. 4B). The energy of the reflected ray is reduced with each collision (e.g. a 30% reduction per collision for bare aluminium), so increasing the number of collisions by promoting diffuse reflection reduces the energy of rays at the second end 20 of the device 10. This means that the ultraviolet radiation can be attenuated by promoting diffuse reflection off the internal walls 22 of the second flow passage portion 26. This can be seen from the lower magnitude of the energy of the reflected ray in FIG. 4B compared to the reflected ray in FIG. 4A. Accordingly, for a given length of the elongate flow passage 16, the energy of the reflected ray is attenuated more effectively when the ray is reflected by diffuse reflection.

The reflection can be made more diffuse by applying a coating to the internal walls 22 of the second flow passage portion 26. As one example, the aluminium material of the internal walls 22 of the second flow passage portion 26 may be coated with black paint (e.g. flat black automotive paint), which reflects incident radiation in a more diffuse manner (i.e. a less specular manner) than bare aluminium. This means that incident radiation can be reflected in an increased range of directions, including back to where it originated, as shown in FIG. 4B. Multiple layers of paint (e.g. at least two coats, or at least three coats) may be used in order to further increase the diffuse nature of the internal walls 22 of the second flow passage portion 26. The black paint may be, for example, Halfords® Matt Black Car Paint, available from Halfords Group Plc, Redditch, UK. This paint reflects between 0% and 5% of incident radiation at ultraviolet wavelengths, as reported in “Common Black Coatings-Reflectance and Ageing Characteristics in the 0.32 μm to 14.3 μm Wavelength Range”, Dury et al, Optics Communications, 270(2):262-272, February 2007, the contents of which are hereby incorporated by reference.

By implementing internal walls 22 that reflect incident radiation in a number of directions (i.e. in a diffuse manner), the number of collisions of a ray of ultraviolet radiation with the internal walls 22 of the second flow passage portion 26 is increased, when compared with specular reflection off the internal walls 22. A coated surface of the internal walls 22 of the second flow passage portion 26 can, therefore, be implemented in conjunction with a smaller aspect ratio, in order to achieve a given attenuation of ultraviolet radiation. This allows the device 10 to be more compact.

The above discussions concerning aspect ratio and the nature of the reflection of incident radiation (i.e. diffuse or specular) assume that a certain percentage of radiation is reflected by the internal walls 22 of the second flow passage portion 26. For example, bare aluminium reflects approximately 70% of incident UVC radiation, which is the material assumed for the examples shown in FIGS. 4A to 4C. In order to reduce the energy of the reflected ray, the internal walls 22 of the second flow passage portion 26 can be configured to reflect a lower proportion of incident UVC radiation. In other words, the internal walls 22 of the second flow passage portion 26 can be configured to absorb a higher proportion of incident UVC radiation. By absorbing a higher proportion of incident UVC radiation, the energy of the rays at the second end 20 of the device 10 is reduced. This means that the ultraviolet radiation can be attenuated by reflecting a lower proportion of incident radiation.

The reflectivity of the internal walls 22 of the second flow passage portion 26 to incident UVC radiation may be reduced by fabricating the honeycomb structure 14 from a material that is less reflective to UVC radiation, or by adding a non-reflective (or less reflective) coating to the material. As one example, the aluminium material of the honeycomb structure 14 (or at least the second flow passage portions 26) may be coated with black paint, which absorbs incident radiation. This means that the energy reflected ray (which is also reflected by diffuse reflection, as described above) is lower. Multiple layers of paint may be used in order to further reduce the reflectivity to UVC radiation.

By configuring the internal walls 22 of the second flow passage portion 26 to reflect a lower proportion of incident UVC radiation than, for example, bare aluminium, the energy of reflected UVC radiation is reduced. This means that internal walls 22 of the second flow passage portion 26 that are less reflective to UVC radiation can be used in conjunction with a smaller aspect ratio, in order to achieve a given attenuation of ultraviolet radiation. This allows the device 10 to be more compact. In order to reduce the energy of reflected radiation, the reflectivity of the internal walls 22 of the second flow passage portion is preferably less than 60% (which provides an improvement over bare aluminium), and more preferably less than 20%, which provides improved performance (i.e. higher attenuation). A reflectivity of about 10% or less (e.g. as provided by flat black automotive paint) can provide further improved performance. A yet further improvement in attenuation reduction can be achieved by configuring the internal walls with a reflectivity of less than 5%.

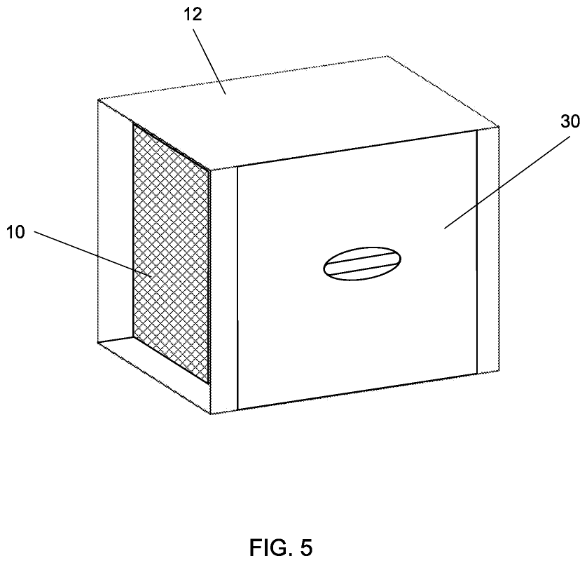

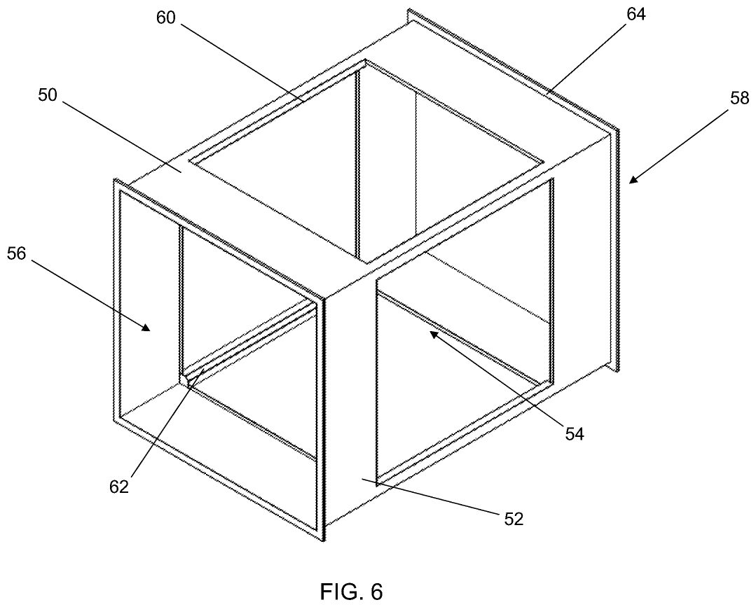

FIG. 5 is a schematic diagram of a ducting section 12 in which the device 10 described with reference to FIGS. 2 to 3B is disposed. The ducting section 12 may, for example, comprise the device 10. As shown in FIG. 5, the ducting section 12 comprises a removable casing 30 that covers an aperture (not shown) in a wall of the ducting section 12. The removable casing 30 may be removed in order to allow access to the device 10 (e.g. for maintenance). The aperture in the ducting section 12 may be large enough to permit the device 10 to be removed from the ducting section 12. In particular, the aperture may have a height that is greater than the height of the device 10, and a width that is greater than the length of the device 10. By allowing removal of the device 10 from the ducting section 12, the device 10 can be removed for maintenance or replacement, for example.

In one particular implementation, the device 10 may be attachable to the removable casing 30 (e.g. removably or permanently attached). This allows an operator to slide the device 10 out of the ducting section 12 using the removable casing 30. Therefore, the device 10 can be removed in a simple manner, without requiring the operator to access the interior of the ducting section 12.

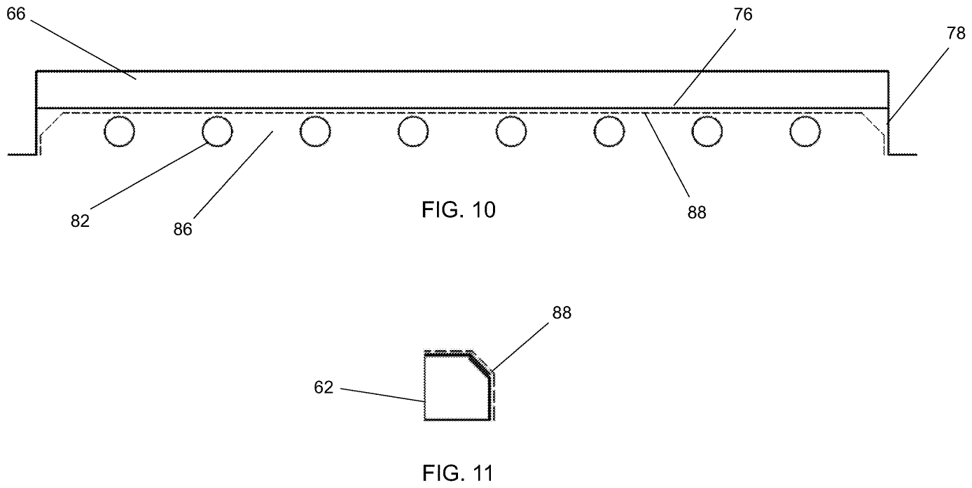

The air cleaning system 40 shown in FIG. 1 comprises a ducting section comprising an inlet and an outlet, such as the ducting section 50 shown in FIG. 6. The air cleaning system 50 also comprises a source of ultraviolet radiation arranged to emit ultraviolet radiation into an interior volume of the ducting section, the interior volume being between the inlet and the outlet. The source of ultraviolet radiation may, for example, be provided in the form of one or more ultraviolet lamps 82 as shown in FIG. 9. The air cleaning system further comprises a reflective surface arranged to reflect ultraviolet radiation emitted by the source of ultraviolet radiation within the interior volume of the ducting section. The reflective surface may be capable of reflecting at least 60% of incident ultraviolet radiation. The reflective surface may, for example, be provided in the form of the layer 88 of reflective material shown schematically in FIG. 10.

In use, the ultraviolet radiation emitted by the source of ultraviolet radiation irradiates any pathogens within the airflow, thereby damaging the RNA or DNA chains of the pathogens and rendering them inactive. The reflective surface reflects the photons emitted by the source of ultraviolet radiation, thereby increasing the energy flux density within the internal volume of the ducting section. In addition, for a determined level of energy flux density to reduce the number of pathogens by a desired proportion, the increased energy flux density provided by the reflective surface can reduce the amount of electrical power needed to power the source of ultraviolet radiation.

FIG. 6 is a top perspective view of a ducting section 50 of the air cleaning system 40. When installed, the ducting section 50 replaces a ducting section of an existing air handling system (such as an existing HVAC system). Therefore, the dimensions of the ducting section 50 are sized to match the dimensions of the ducting section of the existing air handling system that it replaces.

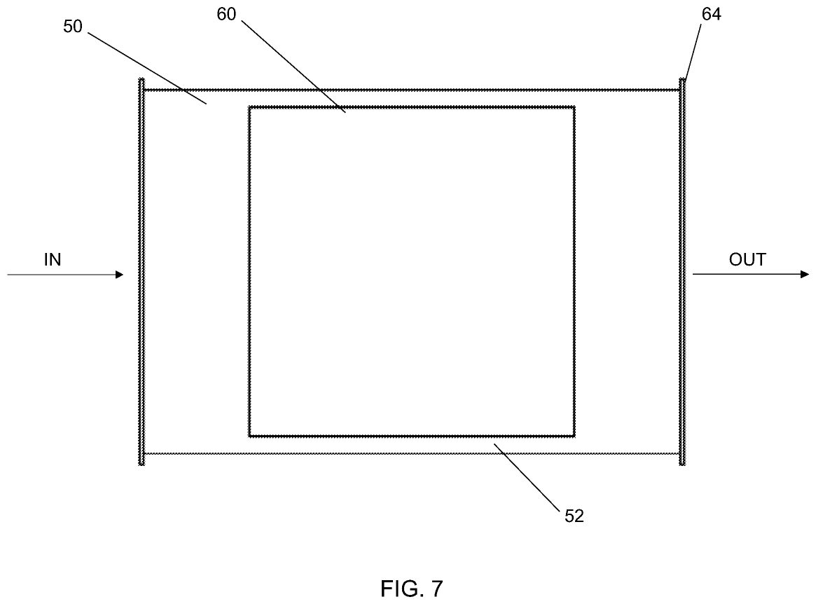

As shown in FIG. 6, the ducting section 50 comprises four walls 52 that define an interior volume 54 of the ducting section 50. As illustrated schematically in FIG. 7, the walls 52 of the ducting section 50 define an air inlet 56 and an air outlet 58. When installed, air flows into the air inlet 56 (e.g. from an upstream adjacent ducting section of the existing air handling system), through the interior volume 54 of the ducting section 50, and out through the air outlet 58 (e.g. to a downstream adjacent ducting section of the existing air handling system).

Each wall 52 comprises an opening 60, meaning that four openings 60 are shown in FIG. 6. The ducting section 50 in the example of FIG. 6 has a square cross-section. In this example, all four openings 60 are the same size, as a result of the consistent dimensions of the walls 52. The skilled person will appreciate that differently-sized openings may be implemented in ducting sections with different cross-sections.

The ducting section 50 further comprises bevelled corner members 62, which extend along the join between two perpendicular walls 52. The bevelled corner members 62 perform two functions. Firstly, they prevent pathogens from travelling along a corner region of the ducting section 50 (which may be subject to a lower level of energy flux density from the ultraviolet radiation source). For this reason, the bevelled corner members 62 are capped along their length and at each end (i.e. the inlet end and the outlet end), thereby forming a hollow member at each corner of the ducting section 50. Secondly, the bevelled corner members 62 provide structural support to the ducting section 50. FIGS. 6 and 7 show that the ducting section 50 also includes two flanges 64, which allow for attachment of the ducting section 50 to adjacent ducting sections of the existing air handling system.

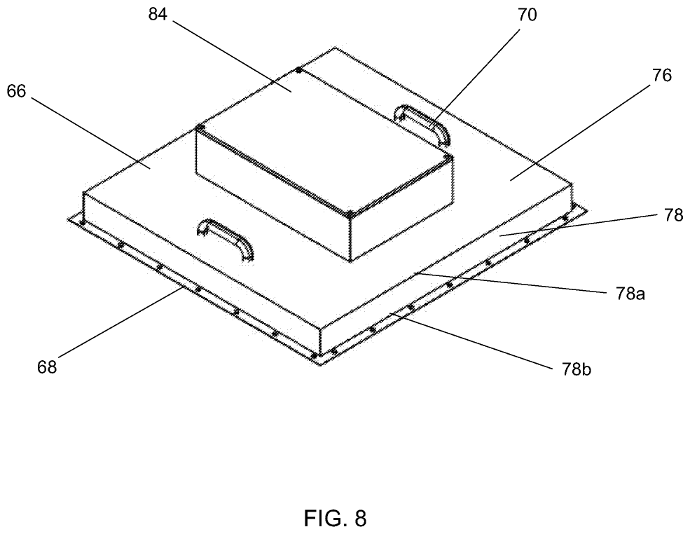

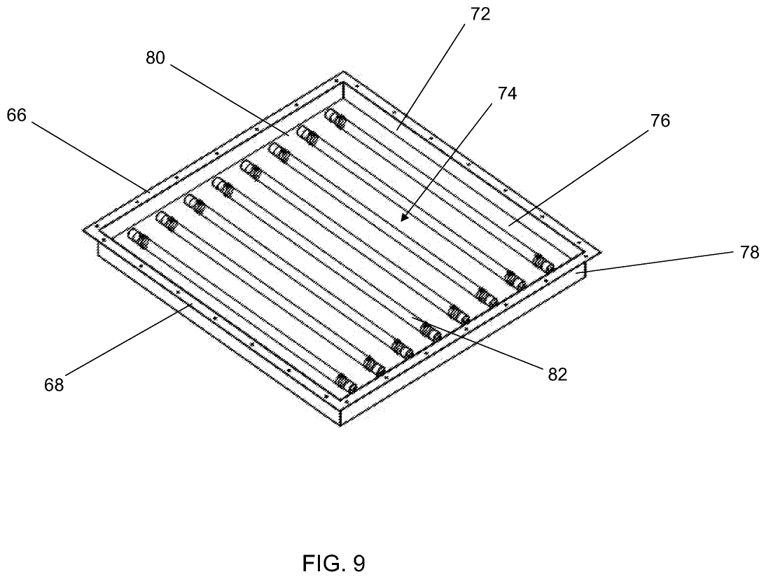

FIG. 8 shows a casing 66 arranged for removable attachment to the ducting section 50. The casing 66 in FIG. 8 is arranged to cover any one of the openings 60 of the ducting section 50 in FIG. 6. For this purpose, the casing 66 shown in FIG. 8 includes a flange 68 for attachment to an exterior surface of a wall 52 of the ducting section 50. The casing 66 also includes handles 70 that allow an operator to manoeuvre the casing 66.

As best shown in FIG. 9, the casing 66 further includes an enclosure 72. The enclosure 72 comprises an interior volume 74 defined by a back wall 76 and four side walls 78 (as best shown in FIG. 8). A first edge 78a of each side wall 78 is attached to the back wall 76. A second edge 78b of each side wall 78 is opposite to the first edge 78a. The second edges 78b of the side walls 78 define an open front face 80 of the enclosure 72.

The handles 70 are attached to an exterior side of the back wall 76 (i.e. opposite to the side of the back wall 76 that defines a surface of the interior volume 74), while the flange 68 is attached to an exterior side of the side walls 78 at the second edges 78b.

One or more sources of ultraviolet radiation (in this example, in the form of ultraviolet lamps 82) are positioned within the enclosure 72 (eight lamps 82 are shown in the example of FIG. 9). Each of the lamps 82 is positioned within the enclosure 72 of the casing 66 so as not to extend beyond a plane of the front face 80 of the enclosure 72. This ensures that the lamps 82 do not protrude into the interior volume 54 defined by the walls 52 of the ducting section 50.

As shown in FIG. 8, the casing 66 further includes an exterior enclosure 84. The exterior enclosure 84 includes power supply components (not shown) for supplying power to the lamps 82. The exterior enclosure 84 also includes circuitry for a safety cut-out switch and negative contact safety switches (not shown), which stop the power to the ultraviolet lamps 82 in the event that the casing 66 is removed from the ducting section 50. The circuitry also includes a connection to the fans that drive the airflow through an air handling system in which the air cleaning system is installed. This prevents an operator from being exposed to an airflow which is potentially contaminated with pathogens, when removing the casing 66. The connection to the fan circuitry is used to turn off the air cleaning system 40 when the HVAC system turns off. The fans also provide airflow over the ultraviolet lamps 82, in order to cool them.

Each of the ultraviolet lamps 82 is arranged to emit ultraviolet radiation. Specifically, each of the ultraviolet lamps 82 is arranged to emit UVC radiation with a wavelength of between about 180 nm and about 280 nm. Preferably, the lamps 82 are arranged to emit UVC radiation with a wavelength of at least 200 nm, in order to avoid production of ozone. A preferred range of UVC radiation is between about 210 nm and about 260 nm. This range includes wavelengths of UVC radiation that are emitted by LEDs. In particularly preferred embodiments, the lamps 82 are arranged to emit UVC radiation with a wavelength of about 222 nm or about 254 nm.

The ultraviolet lamps 82 may comprise any materials that provide for emission of UVC radiation. For example, the ultraviolet lamps 82 may be mercury fluorescent lamps or amalgam lamps. Alternatively, the ultraviolet lamps 82 may be LEDs. As a further alternative, excimer lamps and/or excimer plates may be used as, or in place of, the ultraviolet lamps 82.

FIG. 10 schematically illustrates the arrangement of lamps 82 within the casing 66. As shown in FIG. 10, the lamps 82 are evenly spaced, with gaps 86 between adjacent lamps 82. The combined area of the gaps 86 between the lamps 82 disposed in a particular casing 66 can be expressed as a percentage of the surface area of the back wall 76 of that casing 66 (i.e. an area of a surface on which the lamps 82 are disposed). The combined area of the gaps 86 between the lamps 82 disposed in a particular casing 66 may be between about 50% and about 80% of the surface area of the back wall 76 of the casing 66. In preferred implementations where a smaller number of higher-power lamps 82 are utilised, the combined area of the gaps 86 between the lamps 82 disposed in a particular casing 66 may be between about 70% and about 80% of the surface area of the back wall 76 of the casing 66, particularly preferably between about 75% and about 80% of the surface area.

When viewed from the side of the casing 66 with the lamps 82 visible, a reflective surface (in this example, in the form of a layer 88 of reflective material) is disposed behind the lamps 82. The layer 88 of reflective material therefore extends along the interior surface of the back wall 76, to provide a reflective lining within the interior volume 74 of the enclosure 72. FIG. 10 also shows that the layer 88 of reflective material extends along the interior surfaces of the side walls 78 of the enclosure 72.

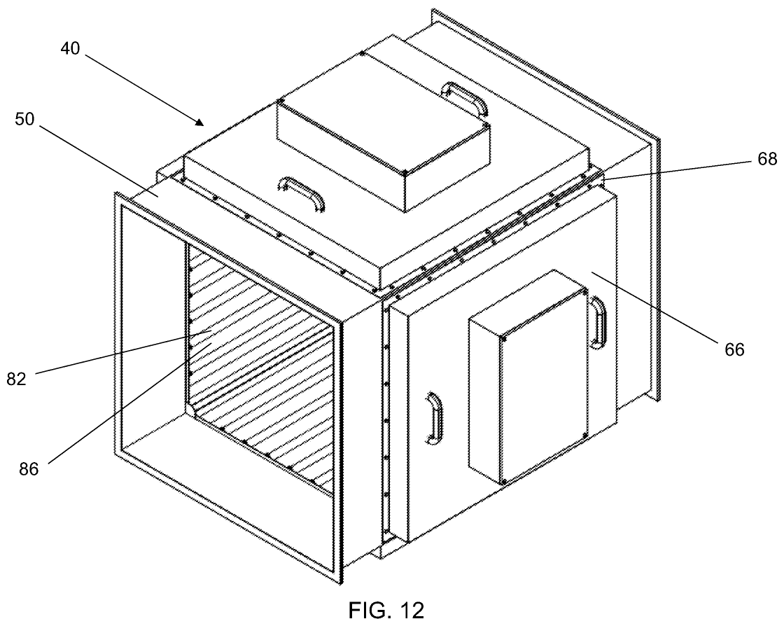

As shown in FIG. 11, the layer 88 of reflective material also extends over the surface of each bevelled corner member 62 that faces the interior volume 54 of the ducting section 50.

The layer 88 of reflective material comprises a material that is capable of reflecting ultraviolet light that is incident on the layer 88. Specifically, the layer 88 comprises a material that reflects UVC radiation without being damaged by the UVC radiation. Preferably, the layer 88 comprises a material that reflects at least 60 percent of the incident UVC radiation. More preferably, the layer 88 comprises a material that reflects at least 65 percent of the incident UVC radiation. More preferably, the layer 88 comprises a material that reflects at least 70 percent of the incident UVC radiation. More preferably, the layer 88 comprises a material that reflects at least 75 percent of the incident UVC radiation. More preferably, the layer 88 comprises a material that reflects at least 80 percent of the incident UVC radiation. More preferably, the layer 88 comprises a material that reflects at least 85 percent of the incident UVC radiation. More preferably, the layer 88 comprises a material that reflects at least 90 percent of the incident UVC radiation. More preferably, the layer 88 comprises a material that reflects at least 95 per cent of the incident UVC radiation. In one example, the layer comprises polytetrafluoroethylene (PTFE), which reflects about 97 percent of incident UVC radiation. In other examples, the layer may comprise nylon, or ultra-high-molecular-weight polyethylene (UHMWPE, also known as UHMW or high-modulus polyethylene (HMPE)), or any combination of these materials. The walls 52 of the ducting section 50 immediately upstream from the openings 60 and immediately downstream from the openings 60 may also be coated with a layer of material that is reflective to UVC radiation, such as PTFE.

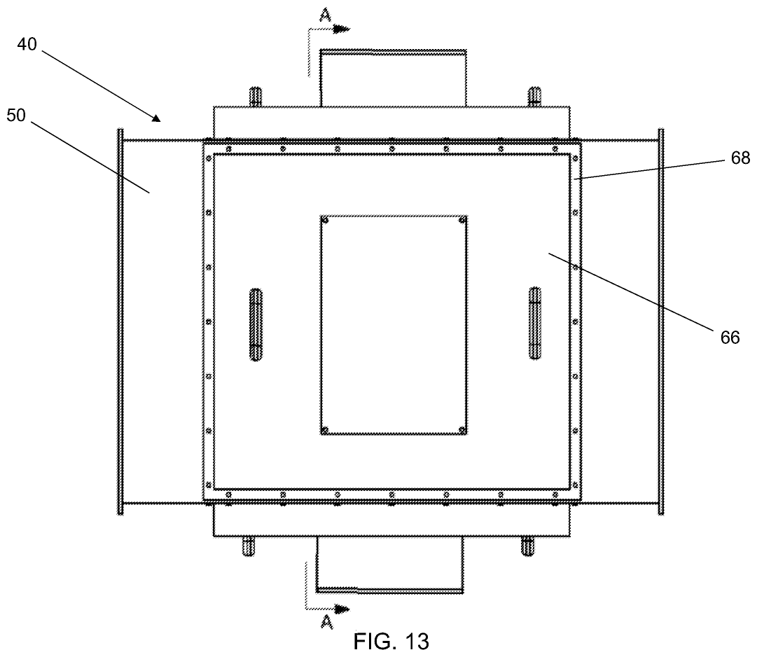

An assembled air cleaning system 40 is shown in FIG. 12. When assembled, each of the openings 52 of the ducting section 50 is covered by a casing 66. In the example shown in FIG. 12, four casings 66 are attached to the ducting section 50. Each casing 66 is attached to the ducting section 50 via screws fitted through holes in the flange 68 of the casing 66 (as seen in FIG. 12 and FIG. 13). Returning to FIG. 12, it can be seen that the casings 66 are attached to the ducting section 50 so that the longitudinal axis of the lamps 82 (i.e. along the length dimension of the lamps) is aligned with the direction of airflow through the ducting section 50 (i.e. a flow path from the air inlet 56 to the air outlet 58). In other words, the longitudinal axis of the lamps 82 is parallel to the direction of airflow through the ducting section 50.

In use, air flows into the interior volume 54 of the ducting section 50 via the air inlet 56. The ultraviolet lamps 82 emit ultraviolet radiation into the interior volume 54 of the ducting section 50. This means that the air flowing through the interior volume 54 is irradiated with ultraviolet (specifically, UVC) radiation from the ultraviolet lamps 82. The assembled air cleaning system 40 comprises four casings 66, each provided to cover an opening 60 in a respective wall 52 of the ducting section 50. This means that the ultraviolet lamps 82 emit ultraviolet radiation into the interior volume 54 from four different directions.

Emission of ultraviolet radiation from multiple different directions increases the irradiation that each virus is exposed to. For example, if ultraviolet radiation is emitted from only one direction, then the ultraviolet radiation “sees” each virus as a circular disc (i.e. as if the virus was present on a surface). Comparatively, if ultraviolet radiation is emitted from two orthogonal directions, then the ultraviolet radiation in each different direction “sees” each virus as a circular disc. However, as the emission directions are different, a greater total surface area of the virus is exposed to the ultraviolet radiation. The skilled person will, of course, appreciate that the virus surface area that is exposed to the ultraviolet radiation may also be increased by irradiating the virus from two non-orthogonal directions (e.g. opposing directions, or directions that are at an angle to one another). In addition, emission of ultraviolet radiation from multiple directions increases the likelihood of irradiating the virus in the event that the virus attaches itself to a larger particle (such as a dust particle). Comparatively, emission of ultraviolet radiation from one direction only (and assuming no reflection of radiation) could result in the larger dust particle providing a barrier to irradiation of the virus.

The layer 88 of reflective material reflects ultraviolet radiation emitted by the ultraviolet lamps 82 within the interior volume 54 of the ducting section 50. This means that the layer 88 of reflective material reflects the UVC radiation emitted by the ultraviolet lamps 82 back into the interior volume 54. In particular, the gaps 86 between the lamps 82 expose the layer 88 of reflective material to the UVC radiation emitted by the lamps 82. This allows the UVC radiation from the lamps 82 to be reflected by the layer 88 of reflective material (rather than being absorbed by the glass of the lamps 82), in order to increase the level of irradiation within the interior volume 54.

In particular, the regions in which the lamps 82 are disposed provide a lower level of reflection of the UVC radiation (around 60% reflectivity), whereas the gaps 86 between the lamps 82 provide higher levels of reflection of UVC radiation owing to the radiation being incident on the layer 88 of reflective material. This higher level of reflection may be above 60% reflectivity (as explained above), and potentially as high as over 95% reflectivity, depending on the material used in the layer 88 of reflective material. Therefore, maximising the combined area of the gaps 86 between the lamps 82 provides an increased area with higher reflectivity. The energy flux density within the air cleaning system 40 can therefore be increased by using a lower number of higher-power ultraviolet lamps 82 and maximising the gaps 86 between the lamps 82.

The energy flux density of the UVC radiation within the interior volume 54 (i.e. by the combination of the lamps 82 and the layer 88 of reflective material) is sufficient to ‘clean’ (or ‘scrub’) the air by inactivating any pathogens in the air flowing through the ducting section 50. The cleaned air then exits the ducting section 50 via the air outlet 58.

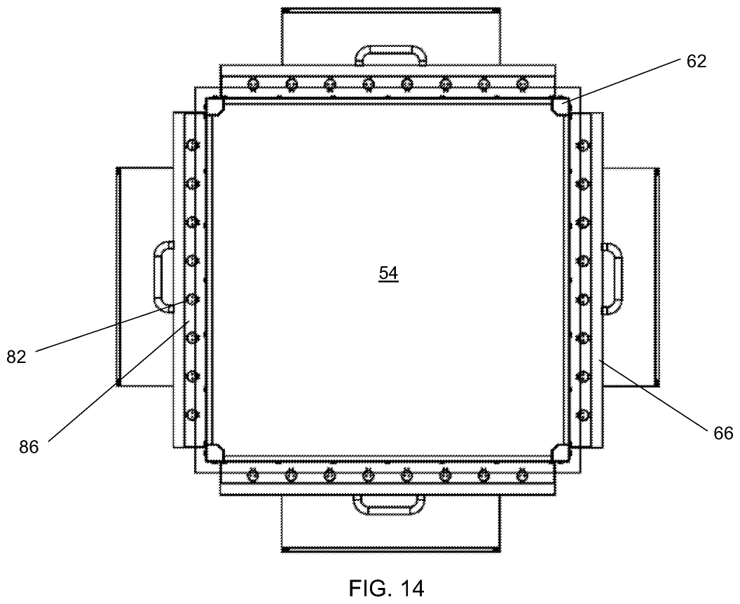

Given that the lamps 82 are disposed within the interior volume 74 of the casing enclosure 72, the lamps 82 do not protrude into the interior volume 54 of the ducting section 50, as best shown in FIG. 14. This mitigates any obstruction of the airflow through the ducting section 50 in use. In other words, the lamps 82 are provided in a recess so as to mitigate obstruction of airflow through the ducting section 50.

The energy flux density within the interior volume 54 of the air cleaning system 40 is sufficient to inactivate 99.99% of SARS-CoV-2 pathogens (i.e. the strain of coronavirus that causes COVID-19) within the air flowing through the ducting section 50. In order to inactivate this percentage of pathogens, the lamps 82 need to provide a particular level of energy flux density. This level of energy flux density is achieved by implementing a particular number of lamps 82 with a certain power.

The determination of the desired energy flux density (and consequently, the number and power of the lamps 82) will now be described.

The fraction of pathogens inactivated on a surface by a particular dose of UVC radiation follows a Poisson distribution which predicts exponential decrease with increasing dosage. This expression can be formulated as:

$$f = exp ( - kD ) \qquad ( Equation 1 )$$

### Where:

f is the remaining fraction of initial pathogens (in other words, 1 minus the inactivation fraction);
D is the dosage of UVC radiation (often given in mJ/cm²); and
k is a constant that varies with the wavelength of the UVC radiation used and the particular pathogen in question (often expressed in cm²/mJ).

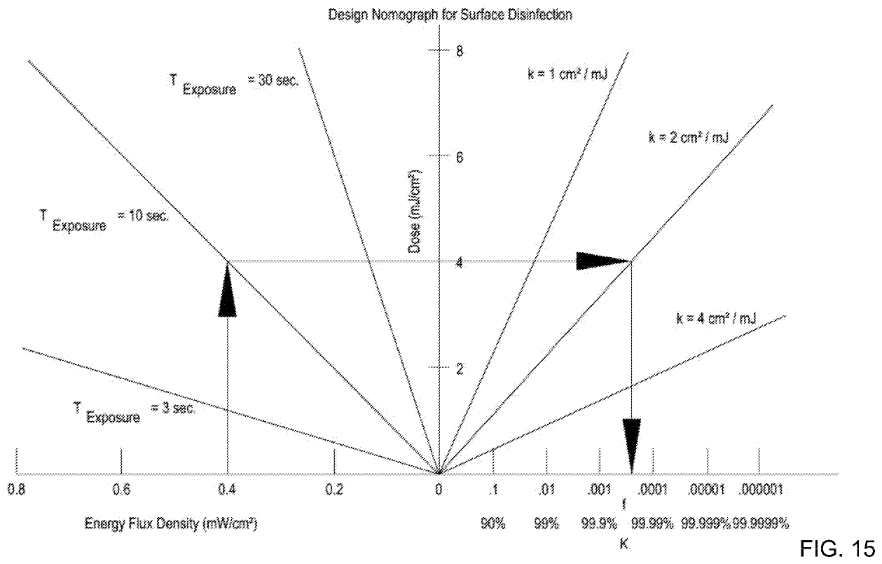

The dosage D in Equation 1 can be calculated as the energy flux density (in mW/cm²) multiplied by the exposure time. FIG. 15 is a nomograph for surface decontamination, showing the pathogen inactivation ratio for an energy flux density of 0.4 mW/cm² illuminating a surface for 10 seconds. The resulting dosage level is 4 mJ/cm². For a pathogen with a k value of 2 cm²/mJ, this dosage level results in an inactivation ratio of about 99.95%.

Equation 1 is typically used for surface irradiation. However, it can also be used in dosage calculations for airborne pathogens within a volume. This is for the reasons explained in the following paragraphs.

SARS-CoV-2 has a diameter of about 100 nm, giving it a cross-sectional area of 7.5×10⁻¹⁵ m². In an example with a duct 1 m across with an aerosol density of one particle per cubic millimetre, with one 100 nm diameter coronavirus per aerosol, the total obscured area of the photon flux will be 10⁹ particles/m³×7.5×10⁻¹⁵ m² virus area×1 virus per aerosol×1 m path length through the air=7.5×10⁻⁵ obscuration fraction. This is 0.0075%, which is low enough to be considered negligible. This means that the beam of photons will propagate through the aerosol cloud undiminished.

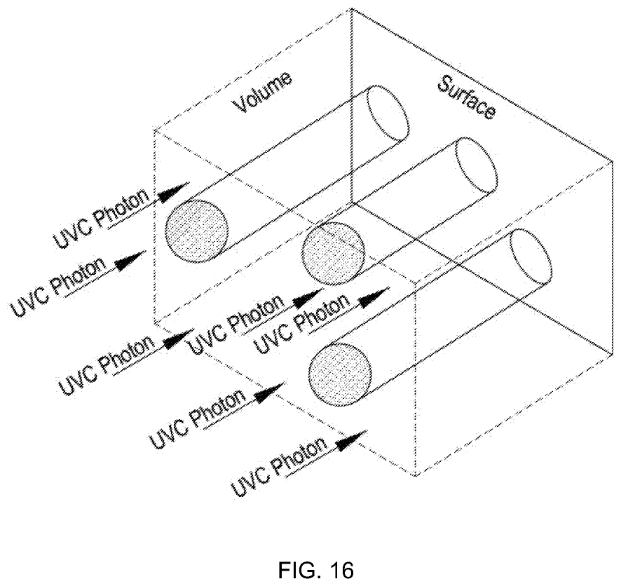

When considering a photon flux traversing virus particles suspended in an airflow, it is useful to imagine the projected area of the particles on a far wall. In light of the very low obscuration fraction (as calculated above), the number of pathogen particles in a typical airflow is far lower than the amount needed to obscure the wall completely. This means that the mortality dynamics can be computed using the exponential law given in Equation 1, but using the surface number density (given by applying f to the number density of particles on a surface—i.e. particles/m²) as the projected density of viruses in aerosols suspended in the air flow. This is illustrated schematically in FIG. 16. As shown in FIG. 16, the total obscured area of the photon beam is very small (as calculated above), meaning that the vast majority of photons are not used. Instead, they will hit the far wall.

Another consideration is the rate of attenuation of an optical beam. This is given by Beer's law, which states that a beam traversing a collection of absorbing particles of cross-sectional area Aₚ and number density n will follow the law:

$$I = {I}_{0} \cdot exp ( - n \cdot {A}_{p} \cdot L ) \qquad ( Equation 2 )$$

### Where:

l is the flux density at a distance L into the medium; and
l₀ is the flux density at the beginning of the medium.

Equation 2 therefore determines the beam attenuation as it traverses the medium. By way of example, the number density of aerosols of 100 nm diameter necessary to attenuate a beam by 50% over a 1 m path can be calculated. The result is about 10¹⁴ particles per cubic metre, or 10⁵ particles per cubic millimetre. It is highly unlikely that particle densities this high would result from human sources (as they would appear as opaque clouds).

The quantity in parentheses in Equation 2 is often referred to as the optical depth or OD (i.e. OD=n·Aₚ·L). If OD<<1, the situation is optically thin. In optically thin situations, the photon interaction with the absorbers is weak, meaning that the beam is not very attenuated. For OD<<1, e^{-OD}=1−OD. In other words, the fraction absorbed is just the OD. This means that the viruses that absorb photons have little effect on the beam intensity. In other words, the beam intensity is substantially constant throughout the cavity (i.e. substantially non-attenuated).

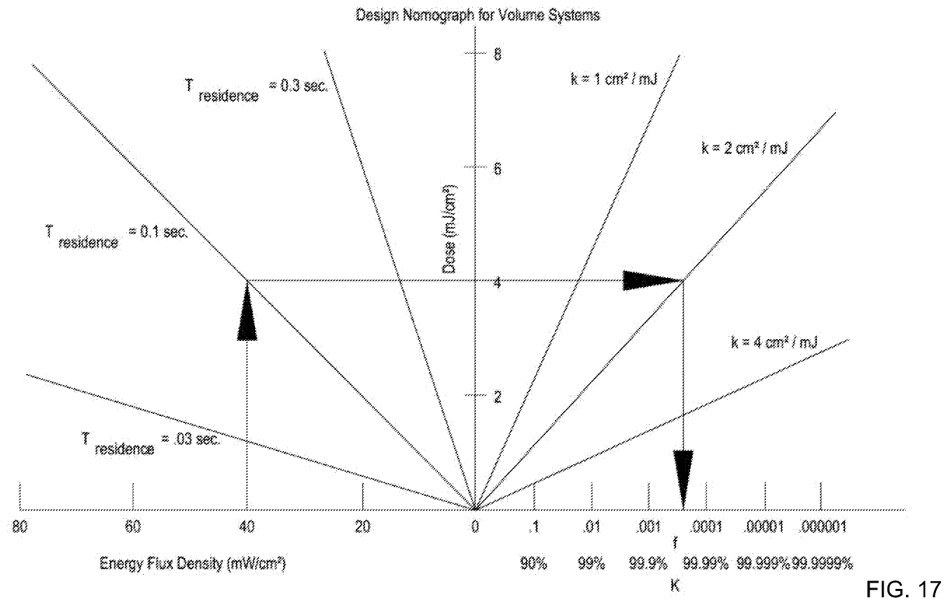

In light of the low obscuration fraction and low beam attenuation, the surface irradiation equation given in Equation 1 can be applied to irradiation of pathogens within a volume of air. For the volume case, the residence time of each particle within the air cleaning system 40 is considered, instead of the exposure time used for the surface irradiation calculation. FIG. 17 is a nomograph for volume irradiation. As shown in FIG. 17, applying an energy flux density of 40 mW/cm² to particles that are resident inside the air cleaning system 40 for 0.1 s (equivalent to a 10 m³/s airflow through a 1 m² duct) provides a dosage of 4 mJ/cm². Again, for a pathogen with a k value of 2 cm²/mJ, this dosage level results in an inactivation ratio of about 99.95% (as with the surface case shown in FIG. 10).

Returning now to Equation 1 and applying it to the specific case of inactivating SARS-CoV-2 pathogens, some values of k for known pathogens and wavelengths of UVC are given in Table 1. These values are taken from existing literature on the use of UVC for disinfection:

TABLE 1
k values for known pathogens and UVC wavelengths.

| Pathogen | Wavelength (nm) | k (cm²/mJ) |
|---|---|---|
| H1N1 | 254 | 2.2-2.9 |
| MRSA | 222 | 2.3 |
| MRSA | 254 | 0.71 |
| H1N1 | 222 | 1.8 |
| H1N1 | 222 | 2.2-2.5 |
| CoV-229E | 222 | 4.1 |
| CoV-OC43 | 222 | 5.9 |
| SARS-CoV-2 | 254 | 1.5-2.2 |
| SARS-CoV-2 | 222 | 2.2-2.3 |

From the data in the above table (in particular, the final two rows), a k value of 2 cm²/mJ can be estimated for both 222 nm and 254 nm photons for inactivating SARS-CoV-2 pathogens.

The total dosage, D, is the energy flux density E″ (in mW/cm²) multiplied by the residence time, t. The residence time, t, can be calculated as:

$$t = L / U \qquad ( Equation 3 )$$

### Where:

L is the length of the internal volume (i.e. the irradiation length); and
U is the velocity of the air flow

The residence time is the time that the air spends inside the air cleaning system 40 (specifically, the time that the air spends within the section of the air cleaning system 40 where it is exposed to ultraviolet radiation from the lamps 82). The velocities used in practical HVAC systems lie within a narrow range. For most existing systems, velocities of the order of 10 m/s are used. Some high speed systems use velocities as high as 15 m/s, but noise considerations and the costs of pumping systems keep velocities of air in ducts within this range. In cases where low noise is desired, velocities can be as little as 3 m/s. In the example calculations below, an air velocity of 10 m/s is used.

Rearranging Equation 1 using Equation 3, we obtain an expression for the energy flux density E″.

$${E}^{″} = - U ln ( f ) / ( k \cdot L ) \qquad ( Equation 4 )$$

As a first example, using f=0.01 (a 99% inactivation ratio), k=2 cm²/mJ, U=10 m/s (as given above) and L=1 m gives E″=23 mW/cm².

For a typical duct of 1 m×1 m cross-section, the volumetric flow rate of air is 10 m³/s. If the UVC photons were emitted and used only once (i.e. not reflected), then the illuminated area would be 1 m² (height×illumination length L), and the total energy of the photons would be 230 W. Assuming a wall plug efficiency of the lamps 82 of 40%, the total electrical power required would be 575 W.

Redoing the above calculation with a desired inactivation ratio of 99.99% (f=0.0001) yields an energy flux density E″=46 mW/cm², requiring a total electrical power of 1151 W assuming 40% lamp efficiency. Doubling the electrical power therefore dramatically increases the inactivation ratio.

The above calculations also assume that UVC photons are used to irradiate the pathogens only once (i.e. they are not reflected). However, as explained in the above example, the interior of the assembled air cleaning system 40 comprises a layer 88 of reflective material such as PTFE.

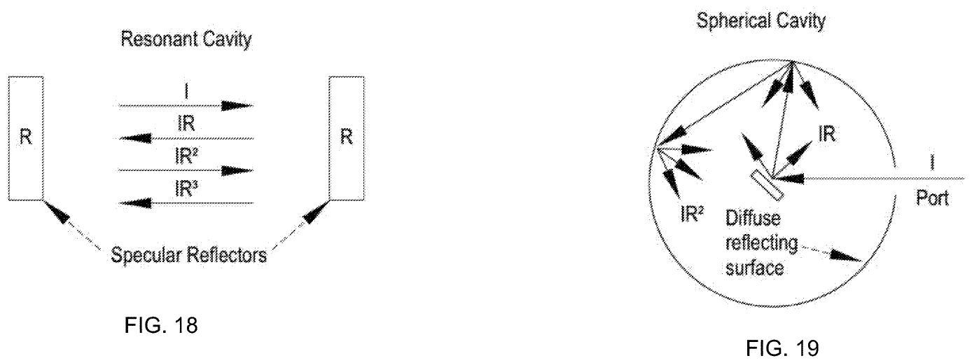

In order to determine the effect of the reflective material, a case with a perfectly collimated beam in between two perfect specular reflectors with finite reflectivity R is considered. This case is illustrated schematically in FIG. 18. On the first pass through the virus cloud, the photon flux density would be I. (The photon flux density can be converted to the energy flux density by considering the energy per photon, Eₚ=(h·c)/A, where h is Planck's constant (6.6×10⁻³⁴ J/s), c is the speed of light (3×10⁸ m/s) and A is the wavelength of the photon in m). The return beam would have photon flux density I·R (neglecting any diffraction effects and assuming that the beam bounces back and forth between the reflectors). The second reflection would yield a beam with flux density I·R². Summing the contributions from the reflected beams would yield a total flux density Iₜₒₜₐₗ=I·(1+R+R²+ . . . +Rⁿ), which simplifies using the theory of series to Iₜₒₜₐₗ=I/(1−R). This means that the multiplier for the cavity with perfect reflectors is 1/(1−R). Inserting some numbers into this equation gives a factor of 10 times the flux density for a 90% reflecting wall, or a factor of 5 times the flux density for an 80% reflecting wall.

Another case that can be considered is an optical cavity in the form of an integrating sphere, illustrated schematically in FIG. 19 as a spherical cavity. Such a device can be used to measure the scattering properties of surfaces. The sphere typically has an interior coated with a very high reflectivity coating. The reflectivity is diffuse and not specular as in the case described in the above paragraph. Access ports allow a light beam to be introduced into one port and directed onto a sample inside. Reflected photons bounce around multiple times and increase the ambient photon flux density. The ratio of increase in photon flux density is given by a similar expression to above, i.e.: M=R/(1−R(1−g)), where g is the fraction of solid angle subtended by all of the ports.

The factor of R in the numerator comes from the fact that the initial beam is not counted in the calculation of the intensity field within the cavity. For the case of the integrating sphere, one of the most important factors in determining the actual multiplier is the loss of photons through the ports.

For a sphere with R=0.9 (i.e. 90% reflectivity) and g=0.33, the multiplier is 2.2 for the cavity. This is expected to be a good approximation of a cavity comprising a cube with two ends open (i.e. the geometry of the air cleaning system 40 described above).

The use of UVC lamps 82 backed by a layer 88 of reflecting material is therefore expected to lead to a significant increase in the energy flux density (and therefore photon flux density) as a consequence of the reflection of photons. PTFE is approximately 97% reflective to UVC photons. Implementing the layer of PTFE is expected to increase the energy flux density by a factor of at least two (potentially as high as three). This can further reduce the total electrical power that needs to be supplied in order to generate the energy flux density necessary to achieve the desired inactivation ratio. For the above example of an inactivation ratio of 99.99%, the total electrical power required to achieve the necessary energy flux density of 46 mW/cm² would reduce to approximately 576 W (i.e. a factor of two), even when ignoring the effects of implementing lamps that emit radiation from all four walls. When combining the effects of reflective material and lamps on all four walls, the total electrical power requirement given above will be reduced even further.

Raytracing programs can be used to compute the actual expected increase in flux density as a consequence of the reflective material, using Monte Carlo techniques.

Returning to FIG. 1, it will be recalled that the air cleaning system 40 is implemented in an air handling system 100 that also comprises a device 10 for reflecting ultraviolet radiation. The device 10 is located downstream of the air cleaning system 40. In particular, the device 10 is located at the outlet 58 of the ducting section 50 of the air cleaning system 40. As shown in FIG. 1, the device 10 is positioned in the air handling system 100 such that the first end 18 of the device 10 is closer to the ultraviolet lamps 82 of the air cleaning system 40 than the second end 20 of the device 10. In this position, the one or more reflective surfaces 28 of the device 10 are configured to reflect ultraviolet radiation from the ultraviolet lamps 82 away from the second end 20 of the device 10 (in other words, towards the internal volume 54 of the air cleaning system 40), as shown schematically in FIG. 4C.

The effect of the device 10 on the energy flux density within the interior volume 54 of the air cleaning system 40 will now be described.

The energy flux density within the interior volume 54 of the air cleaning system can be approximated by the following expression:

$$\frac{E}{A ( 1 - R ( 1 - g ) )} \qquad ( Equation 5 )$$

### Where:

E is the UVC energy input;
A is the total surface area of the interior volume 54;
R is the reflectivity of the interior surfaces of the interior volume 54; and
g is the fraction of the interior surfaces that are not covered with reflective material.

The quantity 1/(1−R(1−g)) is therefore an effective cavity multiplication factor which described how well a cavity (e.g. the air cleaning system 40) amplifies the input flux density E/A.

In an example of a cube with two open ends, in which the walls are coated with a material that is 94% reflective to UVC, g=0.33 because a third of the internal wall area consists of open ends. Inputting g=0.33 and R=0.94 gives a cavity multiplication factor of 2.7.

Extending this example to a hypothetical situation in which the open ends of the cube are configured to reflect the same percentage of radiation back into the cavity as the walls (i.e. 94% incident UVC) gives a value of g of zero, resulting in a cavity multiplication factor of 16.7, which is over six times greater than the open-ended cube example.

From these illustrative examples, the skilled person will appreciate that using the device 10 in combination with the air cleaning system 40 results in a proportion of the photons being reflected back into the interior volume 54 of the air cleaning system 40, thereby increasing the energy flux density within the interior volume 54. These photons would otherwise have exited the air cleaning system 40 via the outlet 58 of the air cleaning system 40. Accordingly, implementing the device 10 in combination with the air cleaning system 40 results in increased energy flux density within the interior volume 54 of the air cleaning system 40, when compared with the energy flux density arising from implementation of the air cleaning system 40 alone.

Increasing the energy flux density within the interior volume 54 of the air cleaning system 40 means that in order to provide the energy flux density associated with a given inactivation ratio, the total electrical power requirement can be decreased. This means that fewer ultraviolet lamps 82 (or lower power ultraviolet lamps 82) can be used within the air cleaning system 40 in order to achieve a given inactivation ratio.

The factor by which the energy flux density within the interior volume 54 is increased depends on a number of variables. A first variable is the length of the first flow passage portion 24. This variable determines whether a photon that collides with an internal wall 22 of a flow passage 16 is reflected (i.e. by the retroreflective surface 28 provided on the internal walls 22 of the first flow passage portion 24) or attenuated (i.e. by the internal walls 22 of the second flow passage portion 26, which may comprise a coating that attenuates incident ultraviolet radiation, as explained above). A second variable is the reflectivity of the material used in the retroreflective surface. For example, aluminium is reflective to over 70% of incident ultraviolet radiation, as explained above.

Returning to FIG. 1, it will be recalled that the device 10 is located between the air cleaning system 40 and the particular location 104 (e.g. a maintenance location) within the air handling system 100. The device 10 attenuates the ultraviolet radiation emitted from within the air cleaning system 40, meaning that the irradiance at the second end 20 of the device 10 is lower than the irradiance at the first end 18 of the device 10. In one example, the device 10 is configured to attenuate the ultraviolet radiation to a level that is safe for a human maintenance worker to carry out maintenance at the particular location 104. In another example, components subject to degradation by ultraviolet radiation are located downstream of the device 10, and the device 10 is configured to attenuate the ultraviolet radiation to a level that prevents or slows down degradation of those components.

It can also be seen from FIG. 1 that the ultraviolet lamps 82 of the air cleaning system 40 are recessed from the interior volume 54 of the air cleaning system 40, in order to mitigate obstruction of airflow within the air cleaning system 40. As the ultraviolet lamps 82 are recessed from the interior volume 54 of the air cleaning system 40, none of the rays from the ultraviolet lamps 82 enters the device 10 in a direction that is parallel to the elongate flow passages 16. Using parameters relating to the recessed positioning of the ultraviolet lamps 82 within the casings 66 and the distance between the ultraviolet lamps 82 and the first end 18 of the device 10, the minimum incidence angle of ultraviolet rays can be calculated. Calculating the minimum incidence angle allows the aspect ratio of the elongate flow passages 16 to be tailored so that ultraviolet rays are unable to pass through the elongate flow passages 16 without colliding with the internal walls 22 of the second flow passage portion 26. In one example, the aspect ratio can be tailored to ensure that ultraviolet rays at the minimum incidence angle collide with the internal walls 22 of the second flow passage portion 26 at least once.

FIG. 20 is a schematic diagram showing an alternative handling system 200, in which two devices 10 for reflecting ultraviolet radiation are implemented. The air handling system 200 comprises an inlet 202 and the airflow direction is indicated by the arrow in FIG. 20. The air handling system 200 includes the air cleaning system 40 shown in FIG. 1. The devices 10 are indicated in FIG. 20 as a first device 10a, located upstream of the air cleaning system 40, and a second device 10b, located downstream of the air cleaning system 40. In particular, the first device 10a is located at the inlet 56 of the ducting section 50 of the air cleaning system 40, while the second device 10b is located at the outlet 58 of the ducting section 50 of the air cleaning system 40.

Specifically, the first device 10a is positioned in the air handling system 200 such that the first end 18 of the first device 10a is closer to the ultraviolet lamps 82 of the air cleaning system 40 than the second end 20 of the first device 10a. In other words, the first device 10a is positioned such that the second end 20 is upstream of the first end 18 within the air handling system 200. In this location, the one or more reflective surfaces 28 of the first device 10a are configured to reflect ultraviolet radiation from the ultraviolet lamps 82 away from the second end 20 of the first device 10a.

Likewise, the second device 10b is positioned in the air handling system 200 such that the first end 18 of the second device 10b is closer to the ultraviolet lamps 82 of the air cleaning system 40 than the second end 20 of the second device 10b. In other words, the second device 10b is positioned such that the second end 20 is downstream of the first end 18 within the air handling system 200. In this location, the one or more reflective surfaces 28 of the second device 10b are configured to reflect ultraviolet radiation from the ultraviolet lamps 82 away from the second end 20 of the second device 10b.

Although not shown in FIG. 20, a first maintenance location may be located upstream of the first device 10a, and a second maintenance location may be located downstream of the second device 10b. The first device 10a and the second device 10b are each configured to reflect ultraviolet radiation into the interior volume 54 of the air cleaning system 40, thereby providing increased energy flux density within the interior volume 54 when compared with the air handling system 100 shown in FIG. 1, and consequently further reducing the electrical power needed to provide the energy flux density associated with the desired inactivation ratio.

The first device 10a also acts to attenuate ultraviolet radiation from the ultraviolet lamps 82 in the air cleaning system 40, meaning that the ultraviolet irradiance upstream of the first device 10a is lower than the irradiance within the air cleaning system 40. Likewise, the second device 10b is also configured to attenuate ultraviolet radiation from the ultraviolet lamps 82 in the air cleaning system 40, meaning that the ultraviolet irradiance downstream of the second device 10b is lower than the irradiance within the air cleaning system 40.

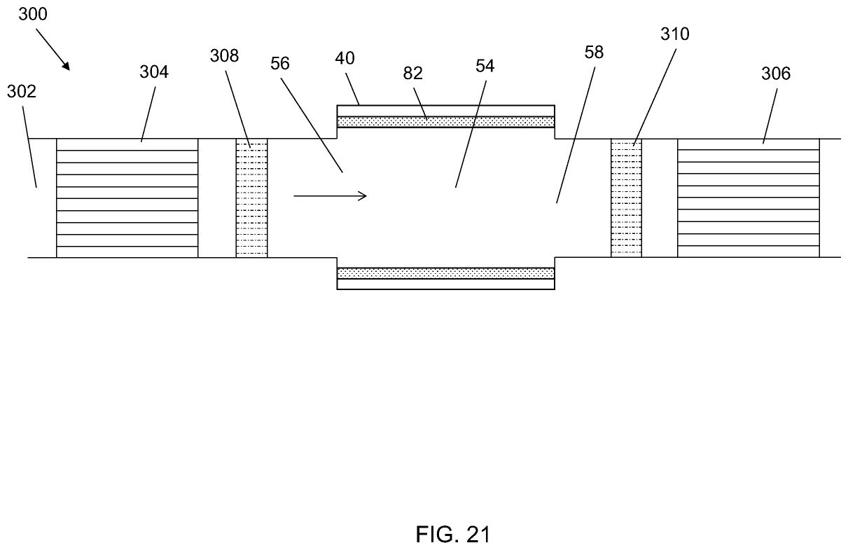

FIG. 21 is a schematic diagram showing a further alternative air handling system 300. The air handling system 300 comprises an inlet 302 and the airflow direction is indicated by the arrow in FIG. 21. The air handling system 300 includes the air cleaning system 40 shown in FIG. 1.

In the system 300 shown in FIG. 21, separate devices are used for reflection and attenuation of ultraviolet radiation. Specifically, a first radiation-attenuating device 304 is located upstream of the inlet 56 of the ducting section 50 of the air cleaning system 40, while a second radiation-attenuating device 306 is located downstream of the outlet 58 of the ducting section 50 of the air cleaning system 40. In addition, a first radiation-reflecting device 308 is located upstream of the inlet 56 of the ducting section 50 of the air cleaning system 40, and downstream of the first radiation-attenuating device 304. Likewise, a second radiation-reflecting device 310 is located downstream of the outlet 58 of the ducting section 50 of the air cleaning system 40, and upstream of the second radiation-attenuating device 306. Each of the radiation-reflecting devices 306, 308 includes a reflective surface, meaning that the reflective surface of each radiation-reflecting devices 306, 308 is disposed between a corresponding radiation-attenuating device 304, 306 and the ultraviolet lamps 82 of the air cleaning device 40.

Each of the radiation-attenuating devices 304, 306 has a construction that is the same as that of the device 10 shown in FIGS. 2 to 4, except that no retroreflective surface is provided on the internal walls of the elongate flow passages. In other words, the internal walls of the elongate flow passages of the radiation-attenuating devices 304, 306 are configured to attenuate ultraviolet radiation that is incident on the internal walls in the same way as the second flow passage portions 26 of the internal walls 22 of the device 10 described above. For example, a desired level of attenuation can be achieved by varying one or more of (i) an aspect ratio of the elongate flow passages; (ii) the nature of the reflection of incident radiation from the internal walls of the elongate flow passages; and (iii) the reflectivity of the internal walls of the elongate flow passages, using the approaches described above with reference to FIGS. 2 to 4.

Each of the radiation-reflecting devices 308, 310 also has a construction that is the same as that of the device 10 shown in FIGS. 2 to 4, except that the retroreflective surface extends along the full length of the internal walls of the elongate flow passages. In other words, the internal walls of the elongate flow passages of the radiation-reflecting devices 308, 310 are configured to reflect ultraviolet radiation that is incident on the internal walls in the same way as the first flow passage portions 24 of the internal walls 22 of the device 10 described above. For example, ultraviolet radiation may be retroreflected from the internal walls by using a retroreflective coating (such as aluminium-coated microspheres) or by embossing the surfaces of the internal walls with a plurality of corner cube reflectors, as described above. The radiation-reflecting devices 308, 310 may be manufactured in the same way as the device 10 described above, except that the full lengths of the elongate flow passages are configured to reflect incident ultraviolet radiation. As one example, therefore, a honeycomb structure of the radiation-reflecting devices 308, 310 may be fully immersed in adhesive before being fully immersed in a container comprising a plurality of aluminium-coated microspheres, in order to provide a retroreflective coating.

The length of the elongate flow passages of the radiation-attenuating devices 304, 306 may be different to (e.g. greater than) the length of the elongate flow passages of the radiation-reflecting devices 308, 310. In other words, the radiation-reflecting devices 308, 310 may be shorter than the radiation-attenuating devices 304, 306, as shown in FIG. 21.

In addition, the diameter of the elongate flow passages of the radiation-reflecting devices 308, 310 may be different to (e.g. smaller than) the diameter of the elongate flow passages of the radiation-attenuating devices 304, 306. FIG. 21 shows an example in which the radiation-reflecting devices 308, 310 have narrower elongate flow passages than the radiation-attenuating devices 304, 306.

The reflective surfaces of the elongate flow passages of the radiation-reflecting devices 308, 310 reflect photons emitted by the ultraviolet lamps 82 back into the interior volume 54 of the air cleaning system 40. As explained above, the ultraviolet lamps 82 of the air cleaning system 40 are recessed from the interior volume 54 of the air cleaning system 40. This means that no photons are emitted from the ultraviolet lamps 82 in a direction that is parallel to the elongate flow passages of the radiation-reflecting devices 308, 310. Using parameters relating to the recessed positioning of the ultraviolet lamps 82 within the casings 66 and the distance between the ultraviolet lamps 82 and the ends of the radiation-reflecting devices 308, 310 that face the interior volume 54, the minimum incidence angle of ultraviolet rays can be calculated.

Calculating the minimum incidence angle allows the aspect ratio of the elongate flow passages of the radiation-reflecting devices 308, 310 to be tailored so that ultraviolet rays are unable to pass through the radiation-reflecting devices 308, 310 without colliding with the internal walls of the elongate flow passages of the radiation-reflecting devices 308, 310. Tailoring the aspect ratio of the elongate flow passages in this way ensures that no photons emitted by the ultraviolet lamps 82 can pass through the radiation-reflecting devices 308, 310 without having previously been reflected at least once (either by the reflective material of the air cleaning system 40, or by the internal walls of the radiation-reflecting devices 308, 310).

The reflection of photons back into the interior volume 54 of the air cleaning system 40 increases the flux density within the interior volume 54, reducing the number and/or power of the ultraviolet lamps 82 required to achieve a given flux density within the interior volume 54. The reflection of photons by the radiation-reflecting devices 308, 310 also means that relatively few photons pass through the radiation-reflecting devices 308, 310. In addition, if the aspect ratio of the elongate flow passages of the radiation-reflecting devices 308, 310 have been configured so that no photons can pass through the radiation-reflecting devices 308, 310 without previously having been reflected, then the energy of the photons passing through the radiation-reflecting devices 308, 310 is attenuated (compared to the energy of non-reflected photons emitted by the ultraviolet lamps 82).

The reduction in number and intensity of photons passing through the radiation-reflecting devices 308, 310 means that the level of attenuation required from the radiation-attenuating devices 304, 306 (e.g. to reduce radiation to a level that is safe for human exposure) is lower than if the radiation-reflecting devices 308, 310 were not present. Consequently, a lower aspect ratio may be used for the radiation-attenuating devices 304, 306 than if the radiation-reflecting devices 308, 310 were not present, because fewer collisions with the internal walls of the radiation-attenuating devices 304, 306 are required in order to reduce the energy of the photons to a desired level.

Using separate radiation-reflecting devices 308, 310 and radiation-attenuating devices 304, 306 also means that the diameter of the flow passages of each radiation-attenuating device 304, 306 is not constrained to the diameter of the flow passages of the radiation-reflecting devices 308, 310. In particular, small diameter flow passages may be used for the radiation-reflecting devices 308, 310, while larger diameter flow passages may be used for the radiation-attenuating devices 304, 306, in order to reduce boundary layer effects on airflow through the elongate flow passages of the radiation-attenuating devices 304, 306. In other words, the diameter of the flow passages of the radiation-attenuating devices 304, 306 are not dictated by the diameter of the flow passages of the radiation-reflecting devices 308, 310.

As a further alternative to using microspheres or corner cube reflectors, the retroreflective material may be provided in the form of a sintered PTFE sheet that is subsequently formed into a honeycomb structure (such as a sintered PTFE sheet available from Porex Corporation of Fairburn, GA, USA). Sintered PTFE is highly reflective to UVC radiation, and is a diffuse reflector. Accordingly, when UVC radiation is incident on the surface of the sintered PTFE honeycomb, a portion of the incident UVC radiation is reflected back towards the source of UVC radiation, meaning that the sintered PTFE acts as a “diffuse retroreflector”. Any photons that are not immediately reflected back continue through the sintered PTFE sheet towards the back of the sheet. As they travel on through the sheet, some photons are reflected back towards the source of UVC radiation. Any photons that pass all the way through the sintered PTFE sheet will continue into an adjacent flow passage of the honeycomb material, where the process is repeated.

In one example, a sintered PTFE sheet may be formed into a honeycomb structure using a kirigami (cutting and folding) method, in the same way as existing kirigami methods that are used for forming paper honeycomb sections. One such existing kirigami method for forming paper honeycomb sections is described in Saito et al., “Manufacture of Arbitrary Cross-Section Composite Honeycomb Cores Based on Origami Techniques”, Journal of Mechanical Design, May 2014, vol. 136, p. 051011, the contents of which are hereby incorporated by reference. The cutting and folding steps used in paper honeycomb manufacture can be applied to a sintered PTFE sheet in order to form a sintered PTFE honeycomb structure.

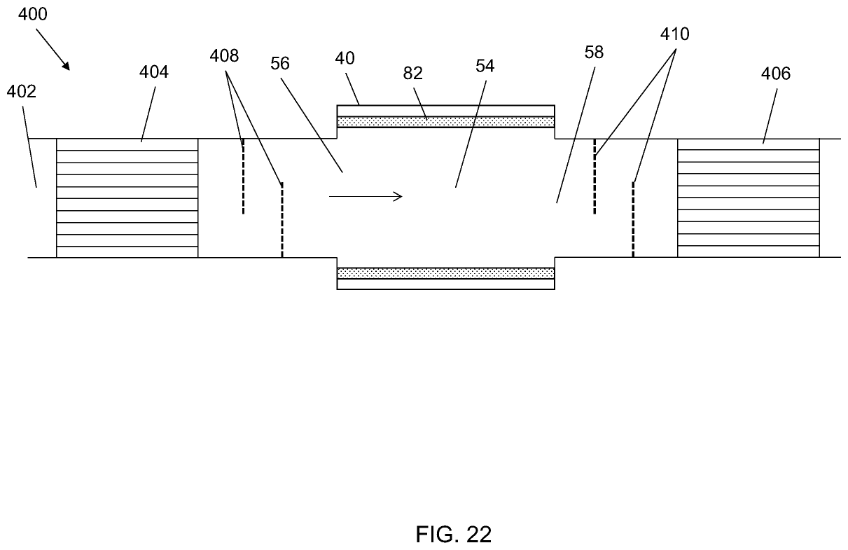

FIG. 22 is a schematic diagram showing a further alternative air handling system 400 in which reflective (optionally retroreflective) baffles are used to reflect UVC photons. In one example, the reflective baffles include a reflective surface formed of a reflective material such as sintered PTFE, which acts as a diffuse retroreflector, as explained above. As with preceding examples, the air handling system 400 includes an inlet 402 and the airflow direction is indicated by the arrow in FIG. 22. The air handling system 400 includes the air cleaning system 40 shown in FIG. 1.

In the system 400 shown in FIG. 22, a first radiation-attenuating device 404 is located upstream of the inlet 56 of the ducting section 50 of the air cleaning system 40, while a second radiation-attenuating device 406 is located downstream of the outlet 58 of the ducting section 50 of the air cleaning system 40. In addition, one or more first radiation-reflecting baffles 408 are located upstream of the inlet 56 of the ducting section 50 of the air cleaning system 40, and downstream of the first radiation-attenuating device 404. Likewise, one or more second radiation-reflecting baffles 410 are located downstream of the outlet 58 of the ducting section 50 of the air cleaning system 40, and upstream of the second radiation-attenuating device 408.

Each of the radiation-attenuating devices 404, 406 has a construction that is the same as that of the radiation-attenuating devices 304, 306 shown in FIG. 21. Each of the radiation-reflecting baffles 408, 410 includes a reflective surface (optionally a retroreflective surface) on a face of the baffle 408, 410 that faces the interior volume 54 of the air cleaning device 40. The reflective surface may be formed of sintered PTFE. The radiation-reflecting baffles 408, 410 are arranged such that any ultraviolet photons travelling orthogonal to a cross-section of the system 400 (i.e. parallel to the arrow) are incident on a reflective surface. In other words, the reflective surfaces of the first radiation-reflecting baffles 408 together cover the entire cross-section of the ducting section, when viewed in a direction opposite to the arrow in FIG. 22, and the reflective surfaces of the second radiation-reflecting baffles 410 together cover the entire cross-section of the ducting section, when viewed in the direction of the arrow in FIG. 22.

Therefore, the reflective surfaces of the radiation-reflecting baffles 408, 410 reflect photons emitted by the ultraviolet lamps 82 back into the interior volume 54 of the air cleaning system 40. The reflection of photons back into the interior volume 54 of the air cleaning system 40 increases the flux density within the interior volume 54, reducing the number and/or power of the ultraviolet lamps 82 required to achieve a given flux density within the interior volume 54.

The reflection of photons by the radiation-reflecting baffles 408, 410 also means that relatively few photons are reflected past the radiation-reflecting baffles 408, 410 in the direction of the radiation-attenuating devices 404, 406. The reduction in number photons reaching the radiation-attenuating devices 404, 406 means that the level of attenuation required from the radiation-attenuating devices 404, 406 (e.g. to reduce radiation to a level that is safe for human exposure) is lower than if the radiation-reflecting baffles 408, 410 were not present. Consequently, a lower aspect ratio may be used for the radiation-attenuating devices 404, 406 than if the radiation-reflecting baffles 408, 410 were not present, because fewer collisions with the internal walls of the radiation-attenuating devices 404, 406 are required in order to reduce the energy of the photons to a desired level. In addition, the increase in flux density within the interior volume 54 may be achieved using a reflective surface that is not necessarily retroreflective, thereby simplifying construction.

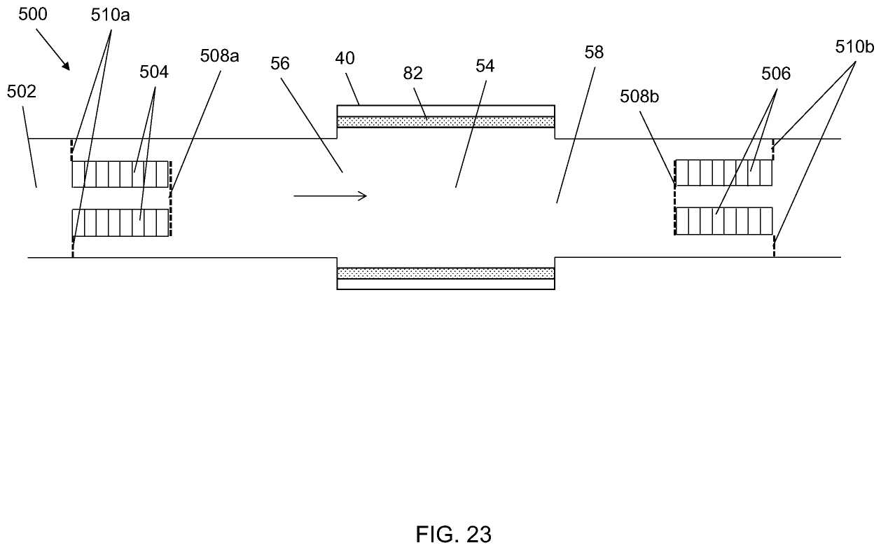

FIG. 23 is a schematic diagram of a further alternative air handling system 500 in which reflective (optionally retroreflective) surfaces are used to reflect UVC photons. In one example, the reflective surfaces are formed of a reflective material such as sintered PTFE. As with preceding examples, the air handling system 500 includes an inlet 502 and the airflow direction is indicated by the arrow in FIG. 23. The air handling system 500 includes the air cleaning system 40 shown in FIG. 1.

In the system 500 shown in FIG. 23, a first pair of radiation-attenuating devices 504 are located upstream of the inlet 56 of the ducting section 50 of the air cleaning system 40, while a second pair of radiation-attenuating devices 506 are located downstream of the outlet 58 of the ducting section 50 of the air cleaning system 40. Each of the radiation-attenuating devices 504, 506 includes flow passages that permit airflow in a direction that is nonparallel to (specifically, substantially perpendicular to) the airflow direction through the air cleaning system 40 (i.e. substantially perpendicular to the direction indicated by the arrow). In the example shown in FIG. 23, the radiation-attenuating devices 504, 506 are orientated such that air enters the radiation-attenuating devices 504, 506 from a volume adjacent to the walls of a ducting section in which the system 500 is implemented, and exits the radiation-attenuating devices 504, 506 into a volume in the centre of the ducting section in which the system 500 is implemented.

The system 500 includes a first radiation-reflecting surface 508a located between the first pair of radiation-attenuating devices 504 and the inlet 56. The first radiation-reflecting surface 508a may be adjacent to an end wall of the each of first pair of radiation-attenuating devices 504, or may form the end wall of each of the first pair of radiation-attenuating devices 504. The first radiation-reflecting surface 508a may also cover the opening between the first pair of radiation-attenuating devices 504, such that air cannot flow between the first pair of radiation-attenuating devices 504 without first flowing through the flow passages of the first pair radiation-attenuating devices 504. This means that the first radiation-reflecting surface 508a acts as a quadrilateral baffle within the system 500. The first radiation-reflecting surface 508a may be formed of a material that is reflective (optionally retroreflective) to UVC radiation, such as sintered PTFE. The system 500 also includes a corresponding second radiation-reflecting surface 508b located between the second pair of radiation attenuating devices 506 and the outlet 58, and having the same construction as the first radiation-reflecting surface 508a.

The system 500 also includes one or more third radiation-reflecting surfaces 510a adjacent to the ends of the first pair of radiation-attenuating devices 504 that are furthest from the inlet 56. In one example, a third radiation-reflecting surface 510a may extend between an end of each radiation-attenuating device 504 furthest from the inlet 56 and a wall of a ducting section in which the system 500 is implemented. In this example, the radiation-attenuating devices 504 may extend across the full width of the ducting section, meaning that the third radiation-reflecting surfaces 510a are quadrilateral surfaces located on either side of the first pair of radiation-attenuating devices 504 (e.g. above and below the first pair of radiation-attenuating devices 504 when referring to the example arrangement shown in FIG. 23). Accordingly, the radiation-reflecting surfaces 508a, 510a are arranged such that any ultraviolet photons travelling orthogonal to a cross-section of the system 500 (i.e. parallel to the arrow) are incident on a reflective surface.

In an alternative example, the radiation-attenuating devices 504 may not extend across the full width of the ducting section, and the third reflecting surface 510a may be a quadrilateral annulus (in which case each radiation-attenuating device 504 may be supported by supports (not shown) extending from the wall of the ducting section). In this case, additional reflective surfaces may be provided to cover the gaps between the first pair of radiation-attenuating devices 504 on either side of the first pair of radiation-attenuating devices 504. These additional reflective surfaces may be provided to prevent air from flowing around the first radiation-reflecting surface 508a (i.e. into or out of the plane shown in FIG. 23) and into the gap between the first pair of radiation-attenuating devices 504.

One or more fourth radiation-reflecting surfaces 510b are also located adjacent to the ends of the second pair of radiation-attenuating devices 506 that are furthest from the outlet 58, and have the same construction as the one or more third radiation-reflecting surfaces 510a.

The radiation-reflecting surfaces 508, 510 reflect photons emitted by the ultraviolet lamps 82 back into the interior volume 54 of the air cleaning system 40, which increases the flux density within the interior volume 54 and thereby reduces the number and/or power of the ultraviolet lamps 82 required to achieve a given flux density. The reflection of photons by the radiation-reflecting surfaces 508, 510 also results in relatively few photons entering the radiation-attenuating devices 504, 506, meaning that the level of attenuation required from the radiation-attenuating devices 504, 506 is lower than if the radiation-reflecting surfaces 508, 510 were not present. Consequently, a lower aspect ratio may be used for the radiation-attenuating devices 504, 506 than if the radiation-reflecting surfaces 508, 510 were not present. In addition, the increase in flux density within the interior volume 54 may be achieved using a reflective surface that is not necessarily retroreflective, thereby simplifying construction. In addition, it can be seen from FIG. 23 that the radiation-reflecting surfaces 508, 510 prevent airflow from bypassing the radiation-attenuating devices 504, 506.

In the example shown in FIG. 23, air enters the radiation-attenuating devices 504, 506 from the periphery of the ducting section in which the system 500 is implemented, and exits the radiation-attenuating devices 504, 506 towards the centre of the ducting section in which the system 500 is implemented. In an alternative arrangement, air may enter the radiation-attenuating devices 504, 506 from the centre of the ducting section and exit towards the periphery of the ducting section. In this alternative case, the first and second reflective surfaces 508 would cover the gap between each pair of radiation-attenuating devices 504, 506 at the ends of the radiation-attenuating devices 504, 506 furthest from the air cleaning system 40, and the one or more third and fourth radiation-reflecting surfaces 510 would prevent airflow around each pair of radiation-attenuating devices 504, 506 at the ends of the radiation-attenuating devices 504, 506 closest to the air cleaning system 40.

Moreover, it will be appreciated that a single radiation-attenuating device 504 may be provided in place of the first pair of radiation-attenuating devices 504, and a single radiation-attenuating device 506 may be provided in place of the second pair of radiation-attenuating devices 506. In this case, each reflective surface 508, 510 would extend between an end of each radiation-attenuating device 504, 506 and a wall of the ducting section in which the system 500 is implemented.

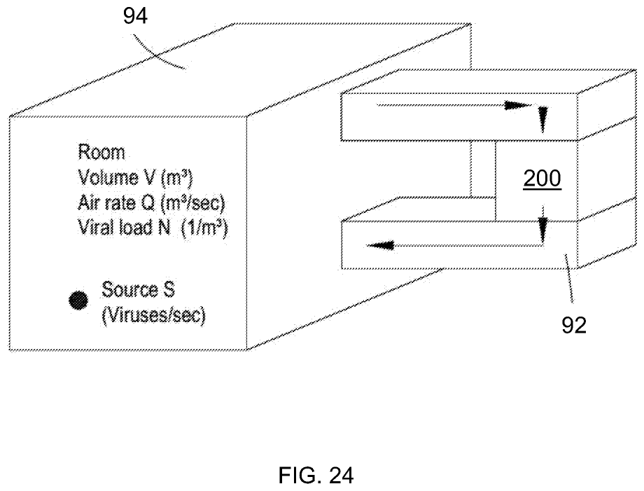

FIG. 24 is a schematic diagram of the air handling system 200 implemented in a HVAC system 92 of a building 94. In an alternative example, any of the air handling systems 100, 300, 400 or 500 may be implemented in the HVAC system 92. A source of viruses that produces S viruses per second is situated in a room with volume V cubic metres. A volumetric flow rate of air of Q m³/s is removed continuously and sent to the air handling system, which includes an air cleaning system such as air cleaning system 40 described above.

The air cleaning system 40 has an inactivation ratio that corresponds to a fractional survival ratio f. Denoting the number of viruses per unit volume in the room in viruses/m³ allows a simple differential equation describing the rate of change of the viral load in the room to be written. This is Equation 6 below. Equation 6 states that the rate of change in the viral load is the difference between the number of viruses that are injected into the room by the source, S, minus the number of viruses that are removed:

$$\frac{{dN}_{v}}{dt} = \frac{S}{V} - \frac{{QN}_{v} ( 1 - f )}{V} \qquad ( Equation 6 )$$

The units of the rate of change of viral load are viruses per cubic metre per second.

Initially, the steady state of the system is considered. In steady state, the viral loading from the source is offset by the reduction of viruses using the air cleaning system 50. This can be investigated by setting the rate of change of viral load (i.e. the left-hand side of Equation 6) to zero. By doing this, we find that:

$${N}_{v} = \frac{S}{Q ( 1 - f )} \qquad ( Equation 7 )$$

From Equation 7, it can be determined that the difference in inactivation ratios is not important in maintaining a low ambient level (provided it is at least 99%), since 1−f is about 1 in either case, whether f=0.01 or 0.0001. However, increasing the amount of air through the system, Q, directly impacts the steady state level.

From a control strategy, if there was a way of measuring the viral loading, then an air handling system comprising the air cleaning system 40 could be run at higher speed (and reduced inactivation ratio) until the levels are lowered. The flow rate could then be lowered to better clean the air (owing to the increased residence time within the irradiation volume of the air cleaning system 40 at lower flow rate). This is an important consideration where the viral load may vary over time.

Returning to Equation 6, we now consider the decay time in the room if the pathogen source leaves. To do this, Equation 6 does not need to be solved. Instead, the terms on the right-hand side of Equation 6 that do not involve N are examined. A decay time can be defined as:

$${τ}_{Decay} = \frac{V}{Q ( 1 - f )} \qquad ( Equation 8 )$$

Substituting Equation 8 into Equation 6 and setting S=0 (i.e. no viral load) gives:

$$\frac{{dN}_{v}}{dt} = \frac{{N}_{v}}{{τ}_{Decay}} \qquad ( Equation 9 )$$

Equation 9 has the solution:

$${N}_{V} = {N}_{V}^{0} {e}^{- t / {τ}_{Decay}} \qquad ( Equation 10 )$$

Equation 10 is a typical exponential decay. Note again that since 1−f is always about 1 regardless of whether the inactivation ratio is 99% or 99.99%, the decay time can simply be estimated as V/Q (which is the turnover time for the room). This is the time required by the system to cycle one room volume through itself.

In one example, a method comprises installing a device 10 as described above for reflecting ultraviolet radiation in an air handling system that comprises a source of ultraviolet radiation.

The air handling system may comprise an air cleaning system 40 as described above (e.g. as with the air handling system 100 or the air handling system 200), in which case the source of ultraviolet radiation is provided in the form of the ultraviolet lamps 82 of the air cleaning system 40. Alternatively, the method may further comprise installing an air cleaning system 40 as described above in the air handling system. In this case, the source of ultraviolet radiation is provided in the form of the ultraviolet lamps 82 of the air cleaning system 40 that is installed in the air handling system. In one example, the air handling system is a HVAC system.

Variations or modifications to the systems and methods described herein are set out in the following paragraphs.

The examples described above relate to a device having elongate flow passages 16 in which a first flow passage portion 24 is retroreflective to incident ultraviolet radiation (e.g. using a retroreflective surface 28) and a second flow passage portion 26 attenuates incident ultraviolet radiation. It will be appreciated that the elongate flow passages 16 may include other flow passage portions, meaning that the first flow passage portion 24 is not necessarily adjacent to the second flow passage portion 26.

In some examples, the first flow passage portion 24 may be long enough that a very high proportion of the ultraviolet radiation is reflected away from the second end 20 of the device 10 by retroreflection off the retroreflective surface 28. In such examples, the proportion of ultraviolet radiation exiting the second end 20 of the device 10 may be low enough that no second flow passage portion 26 is needed. In other words, the device does not need to perform an attenuating function, meaning that an absorbent coating and/or a coating used to promote diffuse reflection can be omitted. In other examples, attenuation of ultraviolet radiation may not be desirable (e.g. if the geometry of a HVAC system prevents photons from reaching a maintenance location), in which case the elongate flow passages 16 may only contain a first flow passage portion 24 having a retroreflective surface 28.

In some examples, the device 10 may be used in conjunction with systems other than the air cleaning system 40. For example, the device 10 may be used in conjunction with a system that uses ultraviolet radiation to clean heating or cooling coils within a HVAC system. The device 10 may therefore be used to increase the flux density in the region of the coils, thereby promoting more efficient cleaning of the coils, or reducing the energy needed to clean the coils. In general terms, therefore, the device 10 may be used in an air handling system (e.g. a HVAC system) that comprises a source of ultraviolet radiation that provides an energy flux density within a particular interior volume of the air handling system. In this case, the device 10 reflects ultraviolet radiation back towards the interior volume of the air handling system. This also applies to the radiation-attenuating devices and the radiation-reflecting devices and surfaces shown in FIGS. 21 to 23.

In examples where a retroreflective surface is formed using microspheres, the microspheres may be formed of any material that is transparent (e.g. glass, silica). A transparent matrix material (e.g. silicone) is also required in order to hold the microspheres in place.

In the above examples, the retroreflective surface 28 is provided on the internal walls 22 of the first flow passage portion 24. It will be appreciated that retroreflection of photons will be achieved even if the retroreflective surface 28 is not provided on all internal walls 22 of the first flow passage portion 24. In particular, it will be appreciated that some retroreflection of photons will be achieved if the retroreflective surface 28 is provided on one or more internal walls 22 of the first flow passage portion 24. Moreover, it will be appreciated that some retroreflection of photons will be achieved if the retroreflective surface 28 is provided on at least one internal wall 22 of at least one elongate flow passage 16. In other words, the retroreflective surface 28 does not need to be provided on the internal wall(s) 22 of each elongate flow passage 16. The same considerations apply to the internal walls of the radiation-reflecting devices 308, 310 shown in FIG. 21.

In other examples, the ultraviolet radiation is reflected by a surface that is not retroreflective. For example, if the elongate flow passages 16 are provided are spaced apart from each other (i.e. not formed using a honeycomb structure 14) such that a surface is provided at the first end 18 of the device 10, then a reflective surface may be provided on the surface at the first end 18 of the device 10, in order to reflect ultraviolet radiation that is incident on the surface at the first end 18 of the device 10. It will be appreciated, however, that spacing the elongate flow passages 16 apart from each other such that a surface is provided at the first end 18 of the device 10 will increase the resistance to air flow through the device 10. The same considerations apply to the radiation-reflecting devices 308, 310 shown in FIG. 21.

In addition, although the elongate flow channels 16 in the above examples have hexagonal cross-sections, it will be appreciated that ultraviolet radiation may be attenuated using other cross-sections of the elongate flow channels 16. For example, the elongate flow channels 16 may have circular, triangular, square, or rectangular cross-sections. In order to minimise the restriction to air flowing through the device 10, it is preferable if the elongate flow channels 16 have cross-sections that can be tessellated (such as triangles, squares, or hexagons). This also applies to the radiation-attenuating devices 304, 306 and the radiation-reflecting devices 308, 310 shown in FIG. 21, and to the radiation-attenuating devices shown in FIGS. 22 and 23.

In addition, although the ducting section 12 in the above examples has a square cross-section, it will be appreciated that the device 10 may be incorporated into a ducting section 12 having a different cross-section. For example, if a HVAC system includes circular ducts, then the device 10 may be incorporated into a ducting section 12 having a circular cross-section. This also applies to the radiation-attenuating devices and the radiation-reflecting devices and surfaces shown in FIGS. 21 to 23.

The skilled person will further appreciate that other colours of paint may be used to reduce the reflectivity of the internal walls 22 and/or to increase the diffuse nature of the reflection off the internal walls 22. For example, although black paint provides optimal absorbance of incident radiation, other dark-coloured paints will also provide reductions in reflectivity of incident radiation. The same considerations apply to the radiation-attenuating devices shown in FIGS. 21 to 23.

The above examples are described with reference to systems in which air flows through the ducting section. The skilled person will appreciate that the above examples are also applicable to systems used for handling gases other than air. In addition, although the above examples are described with particular reference to HVAC systems, the device 10 described herein may also be used in other settings in which leakage of ultraviolet photons is to be minimised without adversely impacting on air or gas flow.

The air cleaning system 40 described above comprises a ducting section 50 with four walls 52. It will be appreciated that the implementations of the air cleaning system 40 described above are also applicable to other duct cross-sections, such as rectangular, triangular or circular cross-sections. In the case of a circular cross section, the ultraviolet lamps 82 may be provided within a casing that is arranged to cover an opening in a cylindrical surface. In this case, the casing will have an arcuate cross-section.

In the above example, the ultraviolet lamps 82 are arranged to emit ultraviolet radiation with a wavelength of 222 nm or 254 nm. It will be appreciated that other wavelengths of ultraviolet radiation may be used to reduce the number of pathogens within a volume of air. Preferably, the wavelength of UVC radiation should be high enough to avoid ozone production but low enough to effectively inactivate the pathogens.

In addition, the ultraviolet lamps 82 in the above example are disposed on all four sides of the duct. In alternative examples, the ultraviolet lamps may not be disposed on all four sides. In particular, the ultraviolet lamps may be disposed on only one of the sides of the duct. If one or more of the sides of the duct are free from ultraviolet lamps, those sides may be coated with a layer of reflective material in order to reflect ultraviolet radiation emitted by the ultraviolet lamps.

Although the above examples include ultraviolet lamps 82 disposed in casings 66 attached to a ducting section 50, the ultraviolet radiation may alternatively be provided by sources of ultraviolet radiation integrated within the walls of a ducting section (which may then not comprise any openings). For example, the ultraviolet radiation may be provided in the form of an ultraviolet light plate which may form part, or all, of the wall of the ducting section. Where sources of ultraviolet radiation are integrated into the walls of the ducting section, the total area of the gaps between the ultraviolet radiation sources (which expose a layer of reflective material to the ultraviolet radiation) may be expressed as a percentage of the surface area of the wall of the ducting section.

Although the above examples are described with reference to reduction of the number of SARS-CoV-2 pathogens, it will be appreciated that the above implementations may also be used to reduce the numbers of different types of pathogens within a volume of air. For example, the above implementations may be used to reduce the numbers of any of the pathogens listed in Table 1, along with influenza, MRSA and tuberculosis. The energy flux density required to reduce the numbers of a specific pathogen may be determined based on the k value for that pathogen. For pathogens with a higher k value than the k value of SARS-CoV-2, the number and power of the ultraviolet lamps may be adapted accordingly.

In order to increase the inactivation fraction provided by the air cleaning system 40, multiple units of the air cleaning system 40 may be provided in series.

The described methods may be implemented using computer executable instructions. A computer program product or computer readable medium may comprise or store the computer executable instructions. The computer program product or computer readable medium may comprise a hard disk drive, a flash memory, a read-only memory (ROM), a CD, a DVD, a cache, a random-access memory (RAM) and/or any other storage media in which information is stored for any duration (e.g., for extended time periods, permanently, brief instances, for temporarily buffering, and/or for caching of the information). A computer program may comprise the computer executable instructions. The computer readable medium may be a tangible or non-transitory computer readable medium. The term “computer readable” encompasses “machine readable”.

The singular terms “a” and “an” should not be taken to mean “one and only one”. Rather, they should be taken to mean “at least one” or “one or more” unless stated otherwise. The word “comprising” and its derivatives including “comprises” and “comprise” include each of the stated features, but does not exclude the inclusion of one or more further features.

The term “retroreflective” is not intended to be limited to reflectors that reflect substantially all incident radiation back at an angle that is substantially equal to the angle of incidence. Instead, the term “retroreflective” is intended to cover diffuse retroreflectors such as sintered PTFE, that reflect a portion of the incident radiation back at an angle that is substantially equal to the angle of incidence, as well as reflecting other portions of the incident radiation in other directions.

The above implementations have been described by way of example only, and the described implementations are to be considered in all respects only as illustrative and not restrictive. It will be appreciated that variations of the described implementations may be made without departing from the scope of the invention. It will also be apparent that there are many variations that have not been described, but that fall within the scope of the appended claims.

The following numbered clauses set out feature combinations that are useful for understanding the present disclosure:

1. A device for reflecting ultraviolet radiation in an air handling system, the device comprising:
a first end and a second end;
a plurality of elongate flow passages, wherein each of the elongate flow passages is configured to permit airflow through the elongate flow passage; and
one or more reflective surfaces configured to reflect incident ultraviolet radiation;
wherein when the device is positioned in the air handling system such that the first end of the device is closer to a source of ultraviolet radiation than the second end of the device, the one or more reflective surfaces are configured to reflect ultraviolet radiation from the ultraviolet radiation source away from the second end of the device.
   2. A device according to clause 1, wherein the one or more reflective surfaces are retroreflective to incident ultraviolet radiation.
   3. A device according to clause 1 or clause 2, wherein each of the plurality of elongate flow passages comprises one or more internal walls.
   4. A device according to clause 3, wherein the one or more reflective surfaces are disposed on at least a portion of at least one of the one or more internal walls of each of the plurality of elongate flow passages.
   5. A device according to clause 4, wherein each of the plurality of elongate flow passages comprises a first flow passage portion and a second flow passage portion, wherein the first flow passage portion of each elongate flow passage is located closer to the first end of the device than the second flow passage portion, and wherein the one or more reflective surfaces are disposed on at least one of the one or more internal walls of the first flow passage portion of each elongate flow passage.
   6. A device according to clause 5, wherein the first flow passage portion of each elongate flow passage is located adjacent to the first end of the device.
   7. A device according to clause 5 or clause 6, wherein the length of the first flow passage portion of each elongate flow passage is between 10% and 40% of the length of the respective elongate flow passage.
   8. A device according to any of clauses 3 to 7, wherein at least a portion of at least one of the one or more internal walls of each of the plurality of elongate flow passages is configured to attenuate incident ultraviolet radiation.
   9. A device according to clause 8, wherein the at least a portion of the at least one of the one or more internal walls of each elongate flow passage comprises a coating configured to absorb a proportion of incident ultraviolet radiation.
   10. A device according to clause 9, wherein the coating is configured to reflect less than 60% of incident UVC radiation, optionally wherein the coating is configured to reflect less than 20% of incident UVC radiation.
   11. A device according to clause 9 or clause 10, wherein the coating comprises one or more coats of black paint.
   12. A device according to any of clauses 8 to 11, when dependent on clause 4, wherein the second flow passage portion of each elongate flow passage is configured to attenuate incident ultraviolet radiation.
   13. A device according to any of clauses 1 to 12, wherein the device is arranged for insertion in a ducting section of the air handling system such that any airflow through the ducting section flows through the elongate flow passages of the device.
   14. A device according to any of clauses 1 to 13, wherein each of the plurality of elongate flow passages is configured to permit air to flow substantially unimpeded through the elongate flow passage.
   15. A device according to any of clauses 1 to 14, wherein each of the plurality of elongate flow passages has an aspect ratio calculated by dividing a length of the elongate flow passage by a diameter of the elongate flow passage, and wherein the aspect ratio of each of the plurality of elongate flow passages is greater than or equal to 4 and less than or equal to 50, optionally wherein the aspect ratio of each of the plurality of elongate flow passages is less than or equal to 20.
   16. An air handling system comprising:
an air cleaning system comprising:
   a ducting section comprising an inlet and an outlet; and
   at least one source of ultraviolet radiation arranged to emit ultraviolet radiation into an interior volume of the ducting section, the interior volume being between the inlet and the outlet; and
a device according to any of clauses 1 to 15, wherein the device is located at one of the inlet and the outlet of the ducting section, wherein the device is configured to reflect ultraviolet radiation towards the interior volume.
   17. An air handling system according to clause 16, wherein the device is a first device, and wherein the air handling system comprises a second device according to any of clauses 1 to 15, wherein the second device is located at the other one of the inlet and the outlet of the ducting section.
   18. An air handling system according to clause 16 or clause 17, wherein the ducting section comprises one or more walls defining a cross-section of the ducting section, wherein the device extends across the entire cross-section of the ducting section, and wherein the at least one source of ultraviolet radiation is recessed from the one or more walls.
   19. An air handling system according to any of clauses 16 to 18, wherein the air cleaning system further comprises a reflective surface arranged to reflect ultraviolet radiation emitted by the source of ultraviolet radiation within the interior volume of the ducting section, wherein the reflective surface is capable of reflecting at least 60% of incident ultraviolet radiation.
   20. An air handling system according to clause 19, wherein the reflective surface comprises a material that is capable of reflecting at least 80% of incident ultraviolet radiation, optionally wherein the material is capable of reflecting at least 90% of incident ultraviolet radiation.
   21. An air handling system according to clause 20, wherein the material comprises one or more of: polytetrafluoroethylene, PTFE, nylon, ultra-high-molecular-weight polyethylene, UHMWPE, or any combination of the foregoing materials.
   22. An air handling system according to any of clauses 19 to 21, wherein the at least one source of ultraviolet radiation comprises a plurality of ultraviolet lamps, wherein adjacent ones of the plurality of ultraviolet lamps are spaced apart from one another to provide a gap between the adjacent ones of the plurality of ultraviolet lamps, and wherein a portion of the reflective surface is exposed to the ultraviolet radiation through the gap between the adjacent ones of the plurality of ultraviolet lamps.
   23. An air handling system according to clause 22, wherein the total area of the gaps is between about 50% and about 80% of an area of a surface on which the ultraviolet lamps are disposed, optionally wherein the total area of the gaps is between about 70% and about 80% of the area of the surface on which the ultraviolet lamps are disposed.
   24. An air handling system comprising:
a source of ultraviolet radiation; and
a device according to any of clauses 1 to 15.
   25. An air handling system according to clause 24, further comprising:
an air cleaning system according to any of clauses 16 to 23, wherein the air cleaning system comprises the source of ultraviolet radiation.
   26. An air handling system according to clause 24 or clause 25, wherein the air handling system is a heating, ventilation and air conditioning, HVAC, system.
   27. A method, comprising:
installing a device according to any of clauses 1 to 15 in an air handling system comprising a source of ultraviolet radiation.
   28. A method according to clause 27, further comprising installing an air cleaning system according to any of clauses 16 to 23 in the air handling system, wherein the air cleaning system comprises the source of ultraviolet radiation.

1. An air handling system, comprising:
at least one source of ultraviolet radiation;
one or more radiation-attenuating devices for attenuating ultraviolet radiation; and
a reflective surface configured to reflect incident ultraviolet radiation away from the one or more radiation-attenuating devices.

2. An air handling system according to claim 1, wherein the reflective surface is disposed between the at least one source of ultraviolet radiation and the one or more radiation-attenuating devices.

3. An air handling system according to claim 1, further comprising a baffle disposed between the at least one source of ultraviolet radiation and the one or more radiation-attenuating devices, and wherein the reflective surface is provided on a face of the baffle.

4. An air handling system according to claim 3, wherein the system comprises a plurality of baffles disposed between the at least one source of ultraviolet radiation and the one or more radiation-attenuating devices, wherein each of the plurality of baffles includes a reflective surface configured to reflect incident ultraviolet radiation away from the one or more radiation-attenuating devices.

5. An air handling system according to claim 1, wherein the system comprises a plurality of reflective surfaces, wherein the plurality of reflective surfaces are arranged such that any ultraviolet photos travelling orthogonal to a cross-section of the air handling system are incident on one or more of the plurality of reflective surfaces.

6. (canceled)

7. An air handling system according to claim 1, wherein the system comprises a plurality of reflective surfaces, wherein the plurality of reflective surfaces are arranged such that one or more of the plurality of reflective surfaces prevent airflow from bypassing the one or more radiation-attenuating devices.

8. An air handling system according to any of claim 1, wherein the reflective surface is retroreflective to incident ultraviolet radiation.

9. An air handling system according to claim 1, wherein the reflective surface is configured to reflect incident UVC radiation away from the one or more radiation-attenuating devices.

10. An air handling system according to claim 1, wherein the air handling system is a heating, ventilation and air conditioning (HVAC) system.

11. An air handling system according to claim 1, wherein each of the one or more radiation-attenuating devices comprises a plurality of elongate flow passages, wherein each of the elongate flow passages is configured to permit airflow through the elongate flow passage.

12. An air handling system according to claim 11, wherein the one or more radiation-attenuating devices are arranged such that any airflow through the air handling system flows through one of the elongate flow passages of one of the one or more radiation-attenuating devices.

13. An air handling system according to claim 11, wherein the one or more radiation-attenuating devices are arranged such that a direction of airflow through the elongate flow passages is nonparallel to a direction of airflow past the at least one source of ultraviolet radiation.

14. An air handling system according to claim 13, wherein the one or more radiation-attenuating devices are arranged such that the direction of airflow through the elongate flow passages is substantially perpendicular to the direction of airflow past the at least one source of ultraviolet radiation.

15. An air handling system according to claim 10, wherein each of the plurality of elongate flow passages comprises one or more internal walls configured to absorb a proportion of incident ultraviolet radiation, wherein the one or more internal walls of each elongate flow passage comprise a coating configured to absorb a proportion of incident ultraviolet radiation.

16. (canceled)

17. An air handling system according to claim 15, wherein the coating is configured to reflect less than 60% of incident UVC radiation, optionally wherein the coating is configured to reflect less than 20% of incident UVC radiation.

18. An air handling system according to claim 15, wherein the coating comprises one or more coats of black paint.

19. A device for reflecting ultraviolet radiation in an air handling system, the device comprising:
a first end and a second end;
a plurality of elongate flow passages, wherein each of the elongate flow passages is configured to permit airflow through the elongate flow passage; and
one or more reflective surfaces configured to reflect incident ultraviolet radiation;
wherein when the device is positioned in the air handling system such that the first end of the device is closer to a source of ultraviolet radiation than the second end of the device, the one or more reflective surfaces are configured to reflect ultraviolet radiation from the ultraviolet radiation source away from the second end of the device.

20. A device according to claim 19, wherein the one or more reflective surfaces are retroreflective to incident ultraviolet radiation.

21. A device according to claim 19, wherein each of the plurality of elongate flow passages comprises one or more internal walls, wherein the one or more reflective surfaces are disposed on at least a portion of at least one of the one or more internal walls of each of the plurality of elongate flow passages.

22. (canceled)

23. A device according to claim 21, wherein each of the plurality of elongate flow passages comprises a first flow passage portion and a second flow passage portion, wherein the first flow passage portion of each elongate flow passage is located closer to the first end of the device than the second flow passage portion, and wherein the one or more reflective surfaces are disposed on at least one of the one or more internal walls of the first flow passage portion of each elongate flow passage, optionally wherein the first flow passage portion of each elongate flow passage is located adjacent to the first end of the device.

24-47. (canceled)
